(19) United States
(12) Patent Application Publication
(10) Pub. No.: US 2026/0232716 A1
(43) Pub. Date: Aug. 13, 2026

(54) NUCLEIC ACID ASSEMBLY PHARMACEUTICAL

(71) Applicants: THE UNIVERSITY OF TOKYO, Tokyo (JP); TKG THERAPEUTICS, INC., Tokyo (JP)

(72) Inventors: Akimitsu OKAMOTO, Tokyo (JP); Kunihiko MORIHIRO, Tokyo (JP); Manami HASEGAWA, Tokyo (JP); Masaaki MATSUI, Tokyo (JP)

(73) Assignees: THE UNIVERSITY OF TOKYO, Tokyo (JP); TKG THERAPEUTICS, INC., Tokyo (JP)

(21) Appl. No.: 19/154,529

(22) PCT Filed: Dec. 19, 2023
(86) PCT No.: PCT/JP2023/045425

§ 371 Date: Aug. 7, 2025

(30) Foreign Application Priority Data
Feb. 7, 2023 (JP) 2023-016897

(51) Int. Cl. A61K 31/7125; A61K 48/00; A61P 35/00; C12N 15/113

(52) U.S. Cl. CPC A61K 31/7125; A61K 48/005; A61P 35/00; C12N 15/113; C12N 2310/31; C12N 2310/531

(57) ABSTRACT

The present invention addresses the problem of providing a composition for treating diseases including cells expressing miR-21, by specifically inducing nucleic acid immunity in cells expressing miR-21. Provided are a cell death-inducing composition including an initiation hairpin nucleic acid and an elongation hairpin nucleic acid, and a pharmaceutical composition and an anticancer agent containing said composition as an active ingredient.

### CROSS-REFERENCE TO RELATED APPLICATIONS

This application is a 371 national phase entry of PCT/JP2023/045425, filed Dec. 19, 2023, which claims the benefit of Japanese Patent Application No. 2023-016897, filed Feb. 7, 2023.

### REFERENCE TO A SEQUENCE LISTING SUBMITTED ELECTRONICALLY

The content of the electronically submitted sequence listing, file name: 522-1294_SequenceListing.xml; size: 66.5 KB; and date of creation: Sep. 3, 2025, is incorporated herein by reference in its entirety.

### TECHNICAL FIELD

The present invention relates to a cell death-inducing composition comprising a hairpin nucleic acid, a pharmaceutical composition comprising it, and an anticancer agent comprising a hairpin nucleic acid.

### BACKGROUND ART

A nucleic acid medicine is a molecular targeted therapeutic drug based on a nucleic acid molecule that specifically binds to a target nucleic acid or protein to suppress its function. Such nucleic acid medicines have been drawing attention as new medicines against diseases that have been conventionally difficult to treat. In fact, in the 2010s, innovative new drugs have been created in Japan and abroad, such as Givlaari, which target the liver as the site of disease (Non-Patent Literature 1). However, the practical application of nucleic acid medicines, particularly against cancer, has not yet been achieved.

Nucleic acid medicines that have been marketed to date have mainly been antisense nucleic acids and siRNAs, which hybridize with target mRNAs in a sequence-specific manner to inhibit the translation and splicing processes of the mRNAs. In recent years, aptamers and decoy nucleic acids that target proteins have also been intensively studied. In particular, the decoy nucleic acids are capable of capturing and/or inactivating nucleic acid-binding proteins, such as transcription factors, for which drug development has been difficult, and are nucleic acid molecules that are promising as anticancer agents. However, there are several problems with the practical application of such decoy nucleic acids as nucleic acid medicines. Primarily, such a decoy nucleic acid has low cell selectivity, and therefore has high off-target effects and is prone to also exhibit toxicity to a normal cell. Further, since the target of a decoy nucleic acid has been limited to a disease for which a specific target protein is present, decoy nucleic acids have not been suitable for the development of medicines that are effective for many diseases.

Medicines targeting microRNAs (miRNAs) are being developed. A miRNA is a non-coding RNA having a length of about 20 to 25 bases, and is known to suppress translation or transcription of a plurality of target genes. In particular, since the miRNA called miR-21 shows high expression in many diseases, including cancers, there is an increasing expectation that miR-21 can be a therapeutic target applicable to a wide range of diseases. However, medicines targeting miR-21 with sufficient cell selectivity have not yet been developed.

In conventional drug development for a nucleic acid medicine, the immunotoxicity exhibited by the nucleic acid medicine itself has been a problem. The immunotoxicity is a consequence of a nucleic acid medicine is recognized as a foreign substance by a defense mechanism against a foreign nucleic acid such as a virus (nucleic acid immunity), and further efforts, such as chemical modification of a nucleic acid or selection of a sequence is required to avoid the immunotoxicity (Non-Patent Literature 2).

Until now, efforts have been made to suppress nucleic acid immunity in nucleic acid medicines. However, induction of nucleic acid immunity against cancer cells and the like in a cell-specific manner may allow the achievement of effective immunotherapy with fewer side effects. Moreover, the achievement may minimize efforts to examine chemical modifications and sequences to suppress nucleic acid immunity, and therefore also provides great advantages in production cost and quality control.

### CITATION LIST

### Non-Patent Literature

Non-Patent Literature 1: Approved Oligonucleotide Therapeutics (May 2021), Section 2, Division of Molecular Target and Gene Therapy Products, National Institute of Health Sciences Non-Patent Literature 2: Shen, W. et al. Nat. Biotechnol. 2019, 37, 640-650.

### SUMMARY OF INVENTION

### Technical Problem

The problem to be solved by the present invention is providing a composition for treating a disease comprising cells expressing miR-21, by inducing nucleic acid immunity specifically in the cells expressing miR-21.

### Solution to Problem

The present inventors previously reported a technique for cell-specific induction of nucleic acid immunity, which technique is applicable to nucleic acid medicines (PCT/JP2022/026323). To solve the problem described above, the present inventors applied the above technique, which had been developed by the inventors themselves, to develop a nucleic acid medicine targeting miR-21. As a result of intensive study of the base sequences of hairpin nucleic acids, the inventors identified components with which a reaction product can be efficiently obtained in a manner specific to the presence of miR-21. The present invention is based on these novel findings and the like, and provides the following.
[1] A cell death-inducing composition comprising a set of hairpin nucleic acids consisting of a starting hairpin nucleic acid comprising a structure represented by general formula (I) and an elongating hairpin nucleic acid comprising a structure represented by general formula (II), wherein the cell death is induced specifically in a cell expressing miR-21.

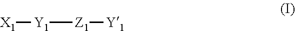

(wherein,
   X₁ is a protruding region consisting of a sequence of 4 to 20 bases;
   Y₁ and Y′₁ are stem regions consisting of sequences of 10 to 20 bases capable of hybridizing with each other intramolecularly;
   Z₁ is a loop region consisting of a sequence of 7 to 12 bases without a CG sequence and a GGG sequence;
   a whole X₁ and a whole or part of Y₁ constitute a miR-21 binding domain capable of hybridizing with a whole or part of the base sequence of miR-21; and
   a whole or part of Y′₁ constitutes an elongating hairpin binding domain capable of hybridizing with a whole or part of miR-21)

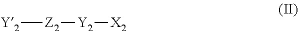

(wherein,
   X₂ is a protruding region consisting of a sequence of 4 to 20 bases;
   Y₂ and Y′₂ are stem regions consisting of sequences of 10 to 20 bases capable of hybridizing with each other intramolecularly;
   Z₂ is a loop region consisting of a sequence of 7 to 12 bases;
   a whole or part of Y′₂ constitutes a miR-21 domain; and
   a whole Y₂ and a whole or part of X₂ constitute a starting hairpin binding domain miR-21 domain capable of hybridizing with a whole or part of the elongating hairpin binding domain)
[2] The cell death-inducing composition according to [1], wherein the left side in the formula (I) and the formula (II) is the 5′ terminus.
[3] The cell death-inducing composition according to [1] or [2], wherein the miR-21 binding domain comprises the base sequence shown in SEQ ID NO: 2, and the miR-21 domain comprises the base sequence shown in SEQ ID NO: 3.
[4] The cell death-inducing composition according to any of [1] to [3], wherein the starting hairpin nucleic acid forms a single-stranded structure in the presence of miR-21 under mammalian physiological conditions.
[5] The cell death-inducing composition according to any of [1] to [4], wherein the GC content of the Z₁ is 40% or more and 60% or less.
[6] The cell death-inducing composition according to any of [1] to [5], wherein the content of purine bases in the Z₁ is 40% or more and 60% or less.
[7] The cell death-inducing composition according to any of [1] to [6], wherein the hairpin nucleic acid is composed of DNA and/or RNA nucleotides.
[8] The cell death-inducing composition according to any of [1] to [7], comprising one or more modified nucleotides and/or unnatural nucleotides.
[9] The cell death-inducing composition according to [8], wherein the modified nucleotide or the unnatural nucleotide comprises a base into which a substituent is introduced.
[10] The cell death-inducing composition according to [8] or [9], wherein the modified nucleotide or the unnatural nucleotide comprises a modified internucleoside bond.
[11] A pharmaceutical composition comprising the cell death-inducing composition according to any of [1] to [10] as an active ingredient.
[12] The pharmaceutical composition according to [11] for preventing or treating cancer or an inflammatory disease.
[13] The pharmaceutical composition according to [12], wherein the cancer is one or more cancers selected from the group consisting of breast cancer, colorectal cancer, pancreatic cancer, lung cancer, prostate cancer, liver cancer, gastric cancer, brain tumor, thyroid cancer, oral cancer, acute myeloid leukemia, chronic lymphocytic leukemia, and glioblastoma.
[14] An anticancer agent for preventing or treating cancer comprising cells expressing miR-21, comprising a set of hairpin nucleic acids consisting of a starting hairpin nucleic acid comprising a structure represented by general formula (I) and an elongating hairpin nucleic acid comprising a structure represented by general formula (II), wherein the cell death is induced specifically in a cell expressing miR-21.

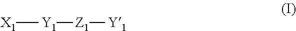

(wherein,
   X₁ is a protruding region consisting of a sequence of 4 to 20 bases;
   Y₁ and Y′₁ are stem regions consisting of sequences of 10 to 20 bases capable of hybridizing with each other intramolecularly;
   Z₁ is a loop region consisting of a sequence of 7 to 12 bases without a CG sequence and a GGG sequence;
   a whole X₁ and a whole or part of Y₁ constitute a miR-21 binding domain capable of hybridizing with a whole or part of the base sequence of miR-21; and
   a whole or part of Y′₁ constitutes an elongating hairpin binding domain capable of hybridizing with a whole or part of miR-21)

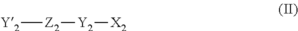

(wherein,
   X₂ is a protruding region consisting of a sequence of 4 to 20 bases;
   Y₂ and Y′₂ are stem regions consisting of sequences of 10 to 20 bases capable of hybridizing with each other intramolecularly;
   Z₂ is a loop region consisting of a sequence of 7 to 12 bases;
   a whole or part of Y′₂ constitutes a miR-21 domain; and
   a whole Y₂ and a whole or part of X₂ constitute a starting hairpin binding domain miR-21 domain capable of hybridizing with a whole or part of the elongating hairpin binding domain)

The contents of the disclosures in Japanese Patent Application No. 2023-016897 which forms the basis for priority of the present application are incorporated herein.

### Advantageous Effects of Invention

According to the cell death-inducing composition of the present invention, cell death can be induced specifically in a cell expressing miR-21.

According to the pharmaceutical composition of the present invention, a disease comprising cells expressing miR-21 can be treated.

According to the anticancer agent of the present invention, a cancer comprising cells expressing miR-21 can be treated.

### BRIEF DESCRIPTION OF DRAWINGS

FIG. 1 is a schematic diagram showing the typical structure of a hairpin nucleic acid of the present invention. FIG. 1A shows the structure of a set of a 5′-end protruding type starting hairpin nucleic acid ((I)) and a 3′-end protruding type elongating hairpin nucleic acid ((II)). FIG. 1B shows the structure of a set of a 3′-end protruding type starting hairpin nucleic acid ((I)) and a 5′-end protruding type elongating hairpin nucleic acid ((II)). In the figure, X represents a protruding region, Y and Y′ represent stem regions, and Z represents a loop region.

FIG. 2 is a diagram that schematically shows a specific example of HCR and the each domain of hairpin nucleic acids. Step 1 to step 3 of HCR are shown.

FIG. 3 is a diagram indicating HCR efficiency in 3 different sets of hairpin nucleic acids (HP-o, HP-a, and HP-b).

FIG. 4 is a diagram indicating HCR efficiency in 2 different sets of hairpin nucleic acids (HP-c and HP-d).

FIG. 5 is a diagram indicating a change in the relative cell survival rate caused by introduction of a set of hairpin nucleic acids in HeLa cells. In the figure, “sc” indicates the result of introduction of hairpin nucleic acids with scrambled sequences. Further, “***” indicates that the p-value is less than 0.001.

### DESCRIPTION OF EMBODIMENTS

### 1. Cell Death-Inducing Composition

### 1-1. Summary

A first aspect of the present invention is a cell death-inducing composition comprising hairpin nucleic acids. The cell death-inducing composition of the present invention comprises a starting hairpin nucleic acid and an elongating hairpin nucleic acid as essential components. According to the cell death-inducing composition of the present invention, cell death can be induced specifically in a cell expressing miR-21, and the composition may be an active ingredient of the pharmaceutical composition of the present invention.

### 1-2. Definitions

The following terms frequently used herein are defined as described below.

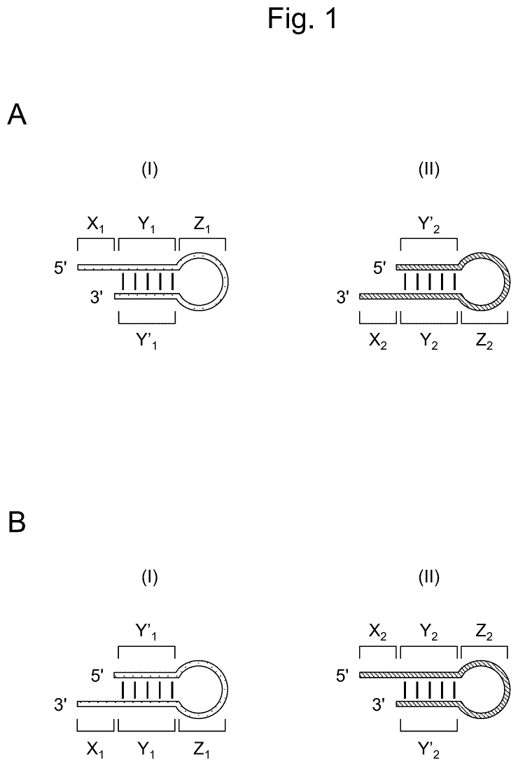

“Hairpin nucleic acid” herein refers to a single-stranded nucleic acid that is capable of forming a hairpin structure. Herein, “hairpin structure” refers to the secondary structure of a nucleic acid comprising a set of a stem structure Y, a loop structure Z, and a protruding region X, formed from a single-stranded nucleic acid. Schematic diagrams of hairpin nucleic acids are shown in FIGS. 1A and 1B. The hairpin nucleic acids herein comprise a protruding region X, a stem region Y, a loop region Z, and a stem region Y′, in this order.

“5′-end protruding types (FIGS. 1A (I) and 1B (II))” herein refer to the types of a hairpin nucleic acid comprising a protruding region X at the 5′-end (5′ terminus). The 5′-end protruding type hairpin nucleic acid comprises a protruding region X, a stem region Y, a loop region Z, and a stem region Y′ in order from the 5′-end. Further, “3′-end protruding types (FIGS. 1A (II) and 1B (I))” herein refer to the types of a hairpin nucleic acid comprising a protruding region X in the 3′-end (3′ terminus). The 3′-end protruding type hairpin nucleic acid comprises a stem region Y′, a loop region Z, a stem region Y, and a protruding region X in order from the 5′-end.

“Stem structure” is a structure in which 2 stem regions (Y and Y′) comprising base sequences capable of hybridizing with each other form a double strand.

“Loop structure” is a loop-shaped structure formed of a loop region (Z) consisting of a single-stranded nucleic acid.

“Protruding region (X)” herein refers to a protruding end that recognizes the single-stranded moiety of miR-21 or the HCR product in HCR. “Protruding end” refers to a nucleic acid region consisting of a single strand adjacent to either or both of the free ends (ends that are not adjacent to loop region Z) of a stem region (Y or Y′). The length of the protruding region X herein is not particularly limited, but is, for example, 4 bases or more, 5 bases or more, 6 bases or more, 7 bases or more, or 8 bases or more. Moreover, the protruding region X may be, for example, 20 bases or less, 19 bases or less, 18 bases or less, 17 bases or less, 16 bases or less, 15 bases or less, 14 bases or less, 13 bases or less, 12 bases or less, 11 bases or less, 10 bases or less, or 9 bases or less. Specifically, the length of the protruding region X may be, for example, 4 to 20 bases.

“Stem regions (Y and Y′)” herein refer to nucleic acid regions that intramolecularly hybridize with each other to form a stem structure. At least both ends of each stem region consist of bases complementary to each other. The length of each stem region is not particularly limited. For example, the length of each stem region is, 10 bases or more, 11 bases or more, 12 bases or more, 13 bases or more, or 14 bases or more, independently from each other. Moreover, the lengths of the stem regions may be, for example, 20 bases or less, 19 bases or less, 18 bases or less, 17 bases or less, 16 bases or less, or 15 bases or less, independently from each other. Specifically, the lengths of the stem regions may be, for example, 10 to 20 bases, independently from each other. “Stem region Y (Y)” refers to a stem region adjacent to the protruding region X. Moreover, “stem region Y′ (Y′)” refers to a stem region that is not adjacent to the protruding region X.

“Loop region (Z)” herein refers to a nucleic acid region located between the 2 stem regions in a single-stranded nucleic acid. The length of the loop region Z is not particularly limited, but is, for example, 7 bases or more, or 8 bases or more. Moreover, the loop region Z may be made up of, for example, 12 bases or less, 11 bases or less, 10 bases or less, or 9 bases or less. Specifically, the length of the loop region Z may be, for example, 7 to 12 bases.

“miR-21” herein refers to a miRNA having the base sequence with a total length of 22 bases shown in SEQ ID NO: 1, as well as a mutant, a homolog, a modified product, and a derivative thereof. miR-21 is known to show increased expression in many cancers, and to be involved in cell growth, cell death, DNA damage response, and the like.

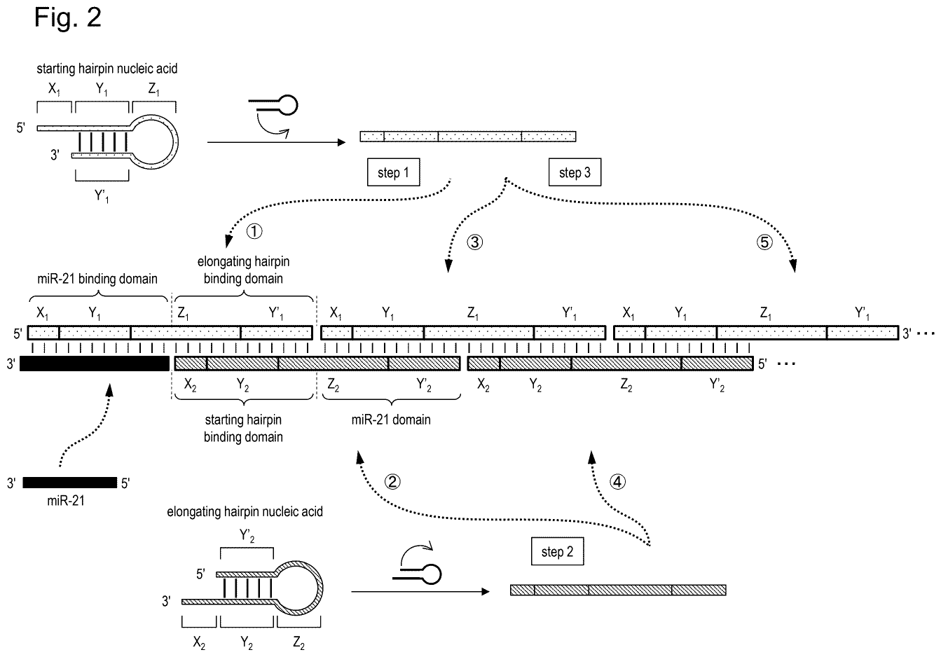

“Hybridization chain reaction (‘HCR’)” herein refers to an elongation reaction of double-stranded nucleic acid molecules, occurring due to hybridization of a plurality of opened (unwound) hairpin nucleic acids serially. A process of typical HCR is schematically shown in FIG. 2. First, in step 1, a starting hairpin nucleic acid recognizes miR-21 as a target with a protruding region X₁, and hybridizes with the miR-21 to form a miR-21 sequence-starting hairpin nucleic acid complex (FIG. 2 (1)). In this step, the hairpin structure of the starting hairpin nucleic acid is opened by elongation of the hybridization with miR-21. The formation of the miR-21 sequence-starting hairpin nucleic acid complex results in the initiation of the formation of an HCR product. Then, in step 2, the protruding region X₂ of an elongating hairpin nucleic acid recognizes the single-stranded moiety (elongating hairpin binding domain) of the starting hairpin nucleic acid in a free state due to the opening, and hybridizes with the single-stranded moiety to form a miR-21 sequence-starting-elongating hairpin nucleic acid complex (FIG. 2 (2)). In this step, the hairpin structure of the elongating hairpin nucleic acid is opened by elongation of the hybridization with the elongating hairpin binding domain of the starting hairpin nucleic acid. In step 3, the starting hairpin nucleic acid recognizes the miR-21-like sequence of the single-stranded moiety (miR-21 domain) of the elongating hairpin nucleic acid in the miR-21 sequence-starting-elongating hairpin nucleic acid complex formed in step 2, is opened, and further hybridizes with the miR-21 sequence-starting-elongating hairpin nucleic acid complex to form a miR-21 sequence-starting-elongating-starting hairpin nucleic acid complex (FIG. 2 (3)). Subsequently, the steps 2 and 3 are alternately repeated to cause sequential hybridization of the starting hairpin nucleic acid and the elongating hairpin nucleic acid, resulting in elongation of the double-stranded nucleic acid to form a macromolecular polymer (HCR product) ((4) and (5) in FIG. 2). The HCR product forms a straight-chain double-stranded nucleic acid.

“Hybridization chain structure” herein refers to a nucleic acid structure comprised in the straight-chain double-stranded nucleic acid formed by the HCR described above. The hybridization chain structure comprises a pathogen-associated molecular pattern, and induces nucleic acid immunity through a pattern-recognition receptor.

“Nucleic acid immunity” herein refers to a natural immune response based on recognition of a nucleic acid. The nucleic acid immunity is induced by recognition of a non-self nucleic acid and/or damaged autologous nucleic acid by a pattern-recognition receptor.

“Pattern-recognition receptor” is a generic term for a receptor protein which is a protein involved in induction of a natural immune system and recognizes a structural pattern seen in a non-self molecule and/or damaged autologous molecule. Herein, unless otherwise specified, the pattern-recognition receptor refers to a receptor protein that recognizes a nucleic acid molecule. A structural pattern recognized by the pattern-recognition receptor is referred to as “pathogen-associated molecular pattern (PAMP)”.

“Pathogen-associated molecular pattern” refers to a molecular pattern that exists in a virus, a prokaryote, and/or a protostome but does not exist in an environment in which a pattern-recognition receptor exists in a normal vertebrate. Herein, “pathogen-associated molecular pattern” particularly refers to a structural pattern that exists in the nucleic acid molecules of a virus, a prokaryote, and/or a protostome but does not exist in the nucleic acid molecule of a vertebrate.

“Complementary” refers to a relationship in which nucleic acid bases can form base pairing with each other through hydrogen bonds. So-called Watson-Crick base pairing (natural type base pairing) or Hoogsteen base pairing is included.

“Hybridize” or “hybridizable” represents that base pairing of polynucleotides having base sequences complementary to each other results in the formation of a completely or partly complementary double strand.

“Plural” herein refers to the number of 2 or more. Specifically, for example, 2 to 60, 2 to 45, 2 to 30, 2 to 14, 2 to 10, 2 to 8, 2 to 6, 2 to 5, 2 to 4, or 2 to 3. “A few” herein refers to the number of 2 to 3.

“Mammalian physiological conditions” herein refers to any conditions that are the same as or similar to the pH and temperature conditions in the body of a mammalian individual. The physiological conditions herein include both in vitro conditions and in vivo conditions. Specifically, the physiological conditions are, for example, conditions in an aqueous buffer at about pH 7.4 (pH 6.8 to pH 7.8) and at about 37° C. (35° C. to 40° C.).

“GC content” herein refers to the ratio of the sum of the numbers of bases of guanine (G) and cytosine (C), to the total number of bases in a base sequence of interest. In particular, when the term is used for the loop region Z₁, it refers to the ratio of the sum of the numbers of bases of G and C, to the total number of bases in the loop region Z₁.

“Purine base” herein refers to a base having a purine skeleton, and broadly encompasses bases including derivatives of purine, which is a heterocyclic compound represented by the chemical formula C₅N₄H₄. Specifically, the purine base includes, for example, adenine (A), guanine (G), hypoxanthine, and xanthine.

“Pyrimidine base” herein refers to a base having a pyrimidine skeleton, and broadly encompasses bases including derivatives of pyrimidine, which is a heterocyclic compound represented by the chemical formula C₄H₄N₂. Specifically, the pyrimidine base includes, for example, cytosine (C), thymine (T), and uracil (U).

“Content of purine bases” herein refers to the ratio of the total number of purine bases to the total number of bases in a base sequence of interest. In particular, when the term is used for the loop region Z₁, it refers to the ratio of the total number of purine bases to the total number of bases in the loop region Z₁.

“Cell death” herein refers to the death of a cell. Such cell deaths are roughly classified into programmed cell deaths and accidental cell deaths. The cell deaths herein include both thereof. The programmed cell death includes, for example, apoptosis, autophagy, and necroptosis. Apoptosis refers to a programmed cell death characterized by formation of an aggregate (apoptotic body) in which a fragmented nucleus is enveloped by a cell membrane. In an apoptosis process, phenomena such as cell fragmentation, nuclear fragmentation, membrane bleb formation, and chromatin condensation are observed. The autophagy herein is also particularly referred to as macroautophagy, and refers to a programmed cell death occurring in nutrient stress. In an autophagy process, phenomena, for example, vacuolation in the wide area of cytoplasm is observed. The necroptosis herein refers to a programmed cell death in which a necrosis-like phenomenon such as extracellular release of a cell content is observed. There are several kinds of programmed cell deaths in which such necrosis-like phenomena are observed, but the necroptosis herein encompasses all of such programmed cell deaths. The accidental cell death refers to a cell death caused by mechanical damage to a cell or by stress in the exterior or interior of a cell. The accidental cell death is also referred to as necrosis.

“High expression” of a specific gene herein refers to a more prominent or significant increase in expression level compared to the expression level in a normal cell.

“Statistically significant” means that when statistically analyzing a difference between the measured value of a subject and a control value, a significant difference between them is observed. For example, cases in which the level of significance (significance level) of an obtained value is low, specifically, less than 5% (p<0.05), less than 1% (p<0.01), or less than 0.1% (p<0.001), are included. Herein, “p (value)” indicates a probability that a test statistic accidentally agrees with the value in a distribution based on the null hypothesis, in a statistical test. Accordingly, less “p” means that the probability that the test statistic agrees with the value is lower, and the null hypothesis is more easily rejected. The testing method is not particularly limited, and a known testing method by which significance can be assessed may be used as a testing method for the statistical analysis, as appropriate. For example, Student's t-test, covariate-variance analysis, and the like may be used.

### 1-3. Components

The cell death-inducing composition of the present invention comprises a set of hairpin nucleic acids.

The set of hairpin nucleic acids consists of a starting hairpin nucleic acid and an elongating hairpin nucleic acid. More specifically, the set of hairpin nucleic acids consists of a 5′-end protruding type starting hairpin nucleic acid and a 3′-end protruding type elongating hairpin nucleic acid, or consists of a 3′-end protruding type starting hairpin nucleic acid and a 5′-end protruding type elongating hairpin nucleic acid. Each of these will be specifically described below.

### (1) Starting Hairpin Nucleic Acid

“Starting hairpin nucleic acid (FIG. 1 (I))” is a hairpin nucleic acid comprising a structure represented by general formula (I), and comprising a miR-21 binding domain and an elongating hairpin binding domain (FIG. 2).

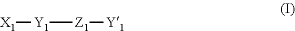

(wherein,
   X₁ is a protruding region consisting of a sequence of 4 to 20 bases;
   Y₁ and Y′₁ are stem regions consisting of sequences of 10 to 20 bases capable of hybridizing with each other intramolecularly;
   Z₁ is a loop region consisting of a sequence of 7 to 12 bases without a CG sequence and a GGG sequence;
   a whole X₁ and a whole or part of Y₁ constitute a miR-21 binding domain capable of hybridizing with a whole or part of the base sequence of miR-21; and
   a whole or part of Y′₁ constitutes an elongating hairpin binding domain capable of hybridizing with a whole or part of miR-21)

As long as the configuration represented by general formula (I) is satisfied, the starting hairpin nucleic acid may additionally comprise another base. For example, a base or base sequence adjacent to the protruding region X₁ and not constituting the miR-21 binding domain may be comprised in the protruding end. Further, a base or base sequence adjacent to Y′₁ and not constituting the stem region Y′₁ and/or the elongating hairpin binding domain may be comprised at the free end of the stem region Y′₁.

“miR-21 binding domain” refers to a nucleic acid region that hybridizes with miR-21, in the starting hairpin nucleic acid. Specifically, for example, the miR-21 binding domain comprises the whole or part of the protruding region X₁, and the whole or part of the subsequent stem region Y₁ (FIG. 2). The miR-21 binding domain is capable of hybridizing with the whole or part of the miR-21 base sequence, and, in the base sequence of this domain, at least both ends of the domain are complementary to miR-21.

“Elongating hairpin binding domain” refers to a nucleic acid region that hybridizes with the elongating hairpin nucleic acid, in the starting hairpin nucleic acid. Specifically, for example, the elongating hairpin binding domain comprises the whole or part of the stem region Y′₁ (FIG. 2). The elongating hairpin binding domain is capable of hybridizing with the starting hairpin binding domain of the elongating hairpin nucleic acid, and the base sequences of the domains are complementary to each other in at least both ends of each of the domains.

For example, regardless of whether the starting hairpin nucleic acid is of the 5′-end protruding type (the starting hairpin nucleic acid whose 5′-end corresponds to the left side in the formula (I)) or of the 3′-end protruding type (the starting hairpin nucleic acid whose 3′-end corresponds to the left side in the formula (I)), the miR-21 binding domain of the starting hairpin nucleic acid may comprise the base sequence represented by SEQ ID NO: 2, or a base sequence comprising the deletion, substitution, or addition of one or several bases in the base sequence represented by SEQ ID NO: 2.

The base sequence of the loop region Z₁ of the starting hairpin nucleic acid herein is not particularly limited as long as it is without a CG sequence and a GGG sequence.

The GC content in the loop region Z₁ of the starting hairpin nucleic acid is not particularly limited. The GC content may be, for example, 0% or more, 10% or more, 20% or more, 25% or more, 30% or more, 34% or more, 35% or more, 36% or more, 40% or more, or 44% or more. Further, the upper limit of the GC content is not particularly limited. For example, the GC content may be 100% or less, 92% or less, 91% or less, 90% or less, 89% or less, 88% or less, 87% or less, 86% or less, 85% or less, 84% or less, 82% or less, 80% or less, 78% or less, 77% or less, 75% or less, 73% or less, 70% or less, 67% or less, 66% or less, 65% or less, 64% or less, 63% or less, 62% or less, 61% or less, 60% or less, 59% or less, or 58% or less. For example, the GC content may be 0% or more and 92% or less, 20% or more and 90% or less, 34% or more and 80% or less, 40% or more and 60% or less, or 44% or more and 58% or less.

The content of purine bases in the loop region Z₁ of the starting hairpin nucleic acid is not particularly limited. The content of purine bases may be, for example, 0% or more, 10% or more, 20% or more, 25% or more, 30% or more, 34% or more, 35% or more, 36% or more, 40% or more, 44% or more. Further, the upper limit of the content of purine bases is not particularly limited. For example, the content of purine bases may be 100% or less, 92% or less, 91% or less, 90% or less, 89% or less, 88% or less, 87% or less, 86% or less, 85% or less, 84% or less, 82% or less, 80% or less, 78% or less, 77% or less, 75% or less, 73% or less, 70% or less, 67% or less, 66% or less, 65% or less, 64% or less, 63% or less, 62% or less, 61% or less, 60% or less, 59% or less, 58% or less, or 57% or less. For example, the content of purine bases may be 0% or more and 92% or less, 20% or more and 90% or less, 34% or more and 80% or less, 40% or more and 60% or less, or 44% or more and 57% or less.

The base sequence of the loop region Z₁ of the starting hairpin nucleic acid may be a sequence in which, for any base sequence consisting of 2 or more consecutive bases, the reverse sequence of its complementary base sequence is not comprised in the same loop region. For example, regarding the base sequence “ATGCATGC”, for the “AT” consisting of the first and second bases from the left end, “AT”, which is the reverse sequence of its complementary base sequence “TA”, is comprised as the 5th to 6th bases. Further, for the “ATG” consisting of the 1st to 3rd bases from the left end, “CAT”, which is the reverse sequence of its complementary base sequence “TAC”, is comprised as the 4th to 6th bases. Therefore, “ATGCATGC” does not correspond to such a base sequence. On the other hand, for example, the base sequence “AGTCAGTC” corresponds to such a base sequence since it does not comprise the reverse sequence of the complementary base sequence of a continuous base sequence regardless of which continuous base sequence is selected. For example, a base sequence comprising a plurality of AT sequences, TA sequences, or GC sequences is not included in such a base sequence.

Further, the loop region Z₁ of the starting hairpin nucleic acid may consist of, for example, any of the following base sequences.
(i) A base sequence comprising 2 or more sequence units of 2 or more consecutive identical bases.
(ii) A base sequence not comprising a sequence of 2 or more consecutive identical bases.
(iii) A base sequence comprising 2 or more sequence units of 3 bases or more that do not comprise identical bases.

Each of the base sequences (i) to (iii) is described below.

### (1-1) Base Sequence (i)

The number of bases comprised in each sequence unit is not particularly limited as long as it is 2 or more. For example, the number of bases may be 3 or more, 4 or more, 5 or more, or 6 or more. The number of bases may be the same or different between sequence units. For example, a first sequence unit may consist of 3 bases, and a second sequence unit may consist of 2 to 4 bases (for example, 2 bases, 3 bases, or 4 bases).

The number of the sequence units is not particularly limited as long as it is 2 or more. For example, the number of the sequence units may be 3 or more, 4 or more, 5 or more, or 6. The types of bases comprised in each sequence unit are not particularly limited. The constituent bases may be the same or different between 2 or more sequence units. For example, 2 or more sequence units consisting of purine bases may be comprised; 2 or more sequence units consisting of pyrimidine bases may be comprised; or a sequence unit(s) consisting of purine bases and a sequence unit(s) consisting of pyrimidine bases may be comprised. When 2 or more sequence units are included, any 2 sequence units preferably do not have sequences consisting of bases complementary to each other. In other words, for example, a sequence unit consisting of Gs and a sequence unit consisting of Cs, or a sequence unit consisting of As and a sequence unit consisting of Ts, are preferably not comprised. Specifically, for example, 2 or more sequence units consisting of Gs, 2 or more sequence units consisting of Cs, 2 or more sequence units consisting of Ts, 2 or more sequence units consisting of As, one or more sequence units consisting of Gs and one or more sequence units consisting of Ts, one or more sequence units consisting of Gs and one or more sequence units consisting of As, one or more sequence units consisting of Cs and one or more sequence units consisting of Ts, or one or more sequence units consisting of Cs and one or more sequence units consisting of As may be comprised.

The number of bases between sequence units is not particularly limited. For example, the sequence units may be adjacent to each other, or one base or more may be comprised therebetween. Specifically, for example, 1 base or more, 2 bases or more, 3 bases or more, 4 bases or more, 5 bases or more, 6 bases or more, 7 bases or more, or 8 bases may be comprised between the sequence units.

The base sequence between the sequence units is not particularly limited as long as the base at each end of the base sequence is different from the adjacent base constituting a sequence unit, and as long as the base sequence does not comprise a sequence of 2 or more consecutive identical bases. Specifically, for example, a base adjacent to a sequence unit consisting of Ts may be other than T. Further, for example, abase adjacent to both a sequence unit consisting of As and a sequence unit consisting of Ts may be other than A or T. For example, a base adjacent to a sequence unit consisting of purine bases may be a purine base, or may be a pyrimidine base.

In particular, when 2 bases or more are comprised between sequence units, the base sequence between the sequence units does not comprise 2 or more consecutive identical bases. For example, the sequence between the sequence units may consist only of bases different from each other. For example, the sequence may comprise both a purine base and a pyrimidine base, or may comprise either a purine base(s) or a pyrimidine base(s) alone. Specifically, for example, when the sequence between the sequence units consists of 2 bases, the sequence may be any of an AC sequence, a CA sequence, a TG sequence, a GT sequence, a GC sequence, an AG sequence, a GA sequence, a TC sequence, a CT sequence, a TA sequence, and an AT sequence.

The bases at both ends of the loop region Z₁ may be bases each constituting a sequence unit, or one or both of the bases may be a base(s) not constituting a sequence unit. Specifically, for example, when the base sequence of the loop region Z₁ is CCCTGAAC, the bases at both ends of the loop region Z₁ are Cs. The C at the left end is a base constituting a sequence unit that is CCC, and the C at the right end is a base not constituting a sequence unit.

The base sequence (i) includes, for example, a base sequence in which the base at each end of the loop region Z₁ is a base constituting a sequence unit, wherein the base sequence comprises one sequence unit consisting of purine bases and one sequence unit consisting of pyrimidine bases, and comprises 2 bases that are different from each other between the 2 sequence units.

Specifically, for example,
the base sequence (i) may comprise
CCTGAA,
CCTGAAA,
CCTGAAAA,
CCCTGAA,
CCCTGAAA,
CCCTGAAAA,
CCCCTGAA,
CCCCTGAAA,
CCCCTGAAAA,
TTGACC,
TTGACCC,
TTGACCCC,
TTTGACC,
TTTGACCC,
TTTGACCCC,
TTTTGACC,
TTTTGACCC,
TTTTGACCCC,
AATGCC,
AATGCCC,
AATGCCCC,
AAATGCC,
AAATGCCC,
AAATGCCCC,
AAAATGCC,
AAAATGCCC,
AAAATGCCCC,
AAGTCC,
AAGTCCC,
AAGTCCCC,
AAAGTCC,
AAAGTCCC,
AAAGTCCCC,
AAAAGTCC,
AAAAGTCCC,
AAAAGTCCCC,
CCAGTT,
CCAGTTT,
CCAGTTTT,
CCCAGTT,
CCCAGTTT,
CCCAGTTTT,
CCCCAGTT,
CCCCAGTTT,
or
CCCCAGTTTT.

### (1-2) Base Sequence (ii)

The base sequence (ii) is not limited as long as it is a base sequence not comprising a sequence of 2 or more consecutive identical bases. For example, the base sequence may be composed by including one or more sequence units of 2 bases or more, which do not comprise identical bases, such that the base sequence does not comprise a sequence of 2 or more consecutive identical bases. In this case, the number of bases comprised in each sequence unit is not particularly limited as long as it is 2 or more. For example, the number of bases may be 3 or more (for example, 3 or 4), or may be, especially when a modified base(s) is/are comprised, 4 or more, for example, 5 or more, or 6 or more. Further, the number of bases may be the same or different between sequence units. For example, a first sequence unit may consist of 3 bases, and a second sequence unit may consist of 3 to 4 bases (for example, 3 bases or 4 bases).

The number of the sequence units is not particularly limited as long as it is one or more. For example, the number of the sequence units may be 2, 3, or 4. The types of the bases comprised in each sequence unit are not particularly limited. Two or more sequence units may have the same base sequence, or may have base sequences that are different from each other. When two or more sequence units are comprised, any two sequence units preferably do not have the reverse complementary relationship (the relationship between a certain base sequence and the reverse sequence of its complementary base sequence) with each other. Specifically, for example, each sequence unit may be a sequence unit consisting of A and T, a sequence unit consisting of A and C, a sequence unit consisting of A and G, a sequence unit consisting of T and C, a sequence unit consisting of T and G, or a sequence unit consisting of a GC sequence, or may be the sequence unit described later for (iii). When 2 or more sequence units have base sequences different from each other, the sequence units may have common constituent bases, or may have constituent bases that are different from each other. Specifically, for example, the sequence units include an AT sequence, a TA sequence, an AC sequence, a CA sequence, an AG sequence, a GA sequence, a TC sequence, a CT sequence, a TG sequence, a GT sequence, a GC sequence, or the sequence units exemplified for the base sequence (iii).

The number of bases between sequence units is not particularly limited. For example, the sequence units may be adjacent to each other, or one base may be comprised therebetween. Usually, when 2 bases are comprised therebetween, the 2 bases constitute another sequence unit.

The base(s) between the sequence units is/are not limited as long as the base(s) is/are selected such that a sequence of 2 or more consecutive identical bases does not appear. In other words, the base(s) is/are not limited as long as each base is different from its adjacent bases. For example, a base adjacent to a sequence unit whose last base is a purine base may be a purine base, or may be a pyrimidine base. Further, a base adjacent to a sequence unit whose first base is a purine base may be a purine base, or may be a pyrimidine base.

### (1-3) Base Sequence (iii)

The base sequence (iii) is not particularly limited as long as it is a base sequence comprising 2 or more sequence units of 3 bases or more that do not comprise identical bases. The number of bases comprised in each sequence unit is not particularly limited as long as it is 3 or more. For example, the number of bases may be 3 or 4, or may be, especially when a modified base(s) is/are comprised, 4 or more, for example, 5 or more, or 6 or more. Further, the number of bases may be the same or different between sequence units. For example, a first sequence unit may consist of 4 bases, and a second sequence unit may consist of 3 to 4 bases (for example, 3 bases or 4 bases).

The number of the sequence units is not particularly limited as long as it is 2 or more. For example, the number of the sequence units may be 2, 3, or 4. The types of the bases comprised in each sequence unit are not particularly limited. Two or more sequence units may have the same base sequence, or may have base sequences that are different from each other. When 2 or more sequence units are included, any 2 sequence units are preferably not sequences complementary to each other. Specifically, for example, each sequence unit may be a sequence unit consisting of A, T, and G, a sequence unit consisting of A, T, and C, a sequence unit consisting of C, T, and G, a sequence unit consisting of A, C, and G, or a sequence unit consisting of A, T, C, and G. When 2 or more sequence units have base sequences different from each other, the sequence units may have common constituent bases, or may have constituent bases that are different from each other. Specifically, for example, the sequence units include an AGT sequence, a GTC sequence, a TCA sequence, a CAG sequence, and reverse sequences thereof (a TGA sequence, a CTG sequence, an ACT sequence, and a GAC sequence); and an AGTC sequence, a GTCA sequence, a TCAG sequence, a CAGT sequence, and reverse sequences thereof (a CTGA sequence, an ACTG sequence, a GACT sequence, and a TGAC sequence).

The number of bases between sequence units is not particularly limited. For example, the sequence units may be adjacent to each other, or 1 base or more may be comprised therebetween. Specifically, for example, 1 base or more, 2 bases or more, 3 bases or more, 4 bases or more, or 5 bases or more may be comprised between the sequence units.

The base sequence between sequence units is not particularly limited. For example, a base adjacent to a sequence unit whose final base is a purine base may be a purine base, or may be a pyrimidine base. Further, a base adjacent to a sequence unit whose first base is a purine base may be a purine base, or may be a pyrimidine base.

When 2 bases or more are comprised between sequence units, the base sequence between the sequence units may or may not comprise a sequence of 2 or more consecutive identical bases. For example, the sequence between the sequence units may consist only of bases different from each other. For example, the sequence may comprise both a purine base and a pyrimidine base, or may comprise either a purine base(s) or a pyrimidine base(s) alone. Specifically, for example, when the sequence between the sequence units consists of 2 bases, the sequence may be any of an AC sequence, a CA sequence, a TG sequence, a GT sequence, a GC sequence, an AG sequence, a GA sequence, a TC sequence, a CT sequence, a TA sequence, and an AT sequence.

The bases at both ends of the loop region Z₁ may be bases each constituting a sequence unit, or one or both of the bases may be a base(s) not constituting a sequence unit. Specifically, for example, when the base sequence of the loop region Z₁ is AGTCAGTA, the bases at both ends of the loop region Z₁ are As. The A at the left end is a base constituting a sequence unit that is AGT, and the A at the right end is a base not constituting a sequence unit.

The base sequence (iii) includes, for example, a base sequence in which the base at least one end of the loop region Z₁ is a base constituting a sequence unit, and which comprises 2 adjacent identical sequence units each consisting of 4 bases.

Specifically, for example, the base sequence (iii) may include AGTCAGTC, GTCAGTCA, TCAGTCAG, CAGTCAGT, or reverse sequences thereof (CTGACTGA, TGACTGAC, GACTGACT, ACTGACTG).

In the starting hairpin nucleic acid, the hairpin structure is preferably opened in the presence of miR-21 under mammalian physiological conditions to form a single-stranded structure. Whether or not the hairpin structure is opened under the physiological conditions may be confirmed using a method known in the art. For example, a cell-free system, an in vitro experimental system using a cell system, an in vivo experimental system, in silico analysis using as an index the free energy change in the hairpin structure formation reaction, or a combination thereof may be employed for the determination. As specific methods of the in vitro experimental system, in vivo experimental system, and in silico analysis that can be used for the determination, for example, the methods exemplified in the Examples of the present application may be used, but the methods are not limited thereto.

“Hairpin structure formation reaction” herein refers to a reaction of a hairpin nucleic acid in which a single-stranded nucleic acid changes from a straight-chain form to a hairpin form to form a hairpin structure.

“Free energy change” herein refers to the net amount of energy supplied from an external environment to a reaction system through a certain reaction under the condition of constant temperature and pressure. “Free energy change” herein particularly corresponds to the net amount of energy supplied from the external environment in the hairpin structure formation reaction. For example, when a reaction product is more thermodynamically stable than a starting material, the reaction system loses energy through the reaction, and therefore, the free energy change is negative. The free energy change of the present invention includes, for example, both Gibbs' free energy change (ΔG) and Helmholtz's free energy change (ΔF).

The free energy change of hairpin structure formation reaction is not particularly limited. Usually, when the value of the free energy change of the hairpin structure formation reaction is lower, a hairpin structure is hardly opened, while a higher value of the free energy change of the hairpin structure formation reaction results in the hairpin structure being more easily opened. The free energy change is, for example, −20 to −10 kcal/mol. Specifically, the free energy change is, for example, −20 kcal/mol or more, −19 kcal/mol or more, −18 kcal/mol or more, −17.5 kcal/mol or more, −17 kcal/mol or more, or −16.5 kcal/mol or more. Moreover, the free energy change is, for example, −10 kcal/mol or less, −11 kcal/mol or less, −12 kcal/mol or less, −12.5 kcal/mol or less, −13 kcal/mol or less, −13.5 kcal/mol or less, −14 kcal/mol or less, −14.5 kcal/mol or less, or −15 kcal/mol or less. The free energy change may be determined using known software that is used in the prediction of the stability of the higher-order structure of a nucleic acid, such as NUPAK.

More specifically, for example, the starting hairpin nucleic acid comprises the following base sequence.
(1) A base sequence represented by any one selected from the group consisting of SEQ ID NOs: 4, 6, 15, and 17
(2) A base sequence comprising the deletion, substitution, or addition of one or a few bases in (1)
(3) A base sequence that hybridizes, under highly stringent conditions, with the base sequence complementary to a base sequence represented by any one selected from the group consisting of SEQ ID NOs: 4, 6, 15, and 17

Hybridizability may be determined using a method known in the art. For example, such hybridizability may be determined based on base identity. Usually, a second nucleic acid is hybridizable with a first nucleic acid when the base identity of the base sequence of the second nucleic acid to the base sequence completely complementary to the base sequence of the first nucleic acid is not less than a certain level. Specifically, for example, when the base identity is 70% or more, 80% or more, 90% or more, 95% or more, 96% or more, 97% or more, 98% or more, 99% or more, or 100%, nucleic acids are hybridizable. “Base identity” herein refers to the percentage (%) of the identical bases of one polynucleotide with respect to the number of all the bases of the other polynucleotide when the base sequences of the two polynucleotides are aligned (subjected to alignment), and, as needed, a gap is introduced into either of the base sequences to allow the degree of the coincidence of the bases of both the base sequences to be the highest. % identity may be easily determined using a known program such as a homology search program BLAST (Basic local alignment search tool, Altschul, S. F. et al, J. Mol. Biol., 215, 403-410, 1990) search. Usually, a second nucleic acid having abase sequence comprising substitution of a plurality of bases with other bases in a base sequence completely complementary to the base sequence of a first nucleic acid is hybridizable with the first nucleic acid. Specifically, for example, when 2 to 60, 2 to 45, 2 to 30, 2 to 14, 2 to 12, 2 to 10, for example, 2 to 8, 2 to 6, 2 to 5, 2 to 4, or 2 to 3 bases are substituted, nucleic acids are hybridizable.

Hybridization conditions are not particularly limited but may be, for example, various stringent conditions such as low and highly stringent conditions. “Low stringent condition” means a condition under which nucleic acids hybridize easily. Such low stringent conditions refer to conditions of low temperature and high salt concentration in washing after hybridization. For example, the low stringent conditions are conditions of performing washing at 42° C. to 50° C. using, for example, a buffer comprising 5×SSC and 0.1% SDS in washing after hybridization. “Highly stringent condition” refers to an environmental condition under which nonspecific hybridization hardly occurs. Under highly stringent conditions, a hybrid can be formed with a nucleic acid having a target base sequence, but a nucleic acid having a nonspecific base sequence cannot substantially form a hybrid. Highly stringent conditions generally refer to conditions of low salt concentration and high temperature. Low salt concentration herein specifically refers to, for example, 15 to 750 mM, preferably, 15 to 500 mM, 15 to 300 mM, or 15 to 200 mM. High temperature herein is, specifically, for example, 50 to 68° C., or 55 to 70° C. Specific examples of highly stringent conditions include conditions of washing at 65° C. and with 0.1×SSC, and 0.1% SDS. Herein, the 1×SSC comprises 150 mM sodium chloride and 15 mM sodium citrate.

### (2) Elongating Hairpin Nucleic Acid

“Elongating hairpin nucleic acid (FIG. 1 (II))” is a hairpin nucleic acid comprising a structure represented by general formula (II), which hairpin nucleic acid hybridizes with the starting hairpin nucleic acid. The elongating hairpin nucleic acid comprises a starting hairpin binding domain and a miR-21 domain.

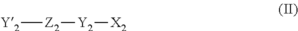

(wherein,
   X₂ is a protruding region consisting of a sequence of 4 to 20 bases;
   Y₂ and Y′₂ are stem regions consisting of sequences of 10 to 20 bases capable of hybridizing with each other intramolecularly;
   Z₂ is a loop region consisting of a sequence of 7 to 12 bases;
   a whole or part of Y′₂ constitutes a miR-21 domain; and
   a whole Y₂ and a whole or part of X₂ constitute a starting hairpin binding domain miR-21 domain capable of hybridizing with a whole or part of the elongating hairpin binding domain)

As long as the conditions represented by general formula (II) are satisfied, the elongating hairpin nucleic acid may additionally comprise another base. For example, a base or base sequence adjacent to the protruding region X₂ and not constituting the starting hairpin binding domain may be comprised in the protruding end. Further, a base or base sequence adjacent to Y′₂ and not constituting the stem region Y′₂ and/or the miR-21 domain may be comprised at the free end of the stem region Y′₂.

“Starting hairpin binding domain” refers to a nucleic acid region that hybridizes with the starting hairpin nucleic acid, in the elongating hairpin nucleic acid. Specifically, for example, the starting hairpin binding domain comprises the whole or part of the protruding region X₂, and the whole or part of the subsequent stem region Y₂ (FIG. 2). The starting hairpin binding domain is capable of hybridizing with the elongating hairpin binding domain of the starting hairpin nucleic acid, and the base sequences of both domains are complementary to each other in at least both ends of each of the domains.

“miR-21 domain” refers to a nucleic acid region having a base sequence capable of hybridizing with a base sequence complementary to the base sequence of miR-21, in the elongating hairpin nucleic acid. Specifically, for example, the miR-21 domain comprises the whole or part of the stem region Y′₂ (FIG. 2). The miR-21 domain is hybridizable with the miR-21 binding domain of the starting hairpin nucleic acid, and the base sequences of both domains are complementary to each other in at least both ends of each of the domains.

For example, regardless of whether the elongating hairpin nucleic acid is of the 3′-end protruding type (the starting hairpin nucleic acid whose 5′-end corresponds to the left side in the formula (II)) or of the 5′-end protruding type (the starting hairpin nucleic acid whose 3′-end corresponds to the left side in the formula (II)), the base sequence of the miR-21 domain of the elongating hairpin nucleic acid may comprise the base sequence represented by SEQ ID NO: 3, or a base sequence comprising the deletion, substitution, or addition of one or several bases in the base sequence represented by SEQ ID NO: 3.

In the elongating hairpin nucleic acid, the hairpin structure is preferably opened in the presence of the starting hairpin nucleic acid under mammalian physiological conditions to form a single-stranded structure.

More specifically, for example, the elongating hairpin nucleic acid comprises the following base sequence.
(1) A base sequence represented by any one selected from the group consisting of SEQ ID NOs: 5, 7, 16, and 18
(2) Abase sequence comprising the deletion, substitution, or addition of one or several bases in (1)
(3) A base sequence that hybridizes, under highly stringent conditions, with the base sequence complementary to a base sequence represented by any one selected from the group consisting of SEQ ID NOs: 5, 7, 16, and 18

The determination of whether or not the single-stranded structure has been formed, the free energy change, the base sequence that hybridizes under highly stringent conditions, and the like are in accordance with those described for the starting hairpin nucleic acid.

The base sequence of miR-21 and the base sequences described above for the starting hairpin nucleic acid and the elongating hairpin nucleic acid are shown in Table 1.

TABLE 1
Base sequences of miR-21 and hairpin nucleic acids

| | | SEQ ID |
|---|---|---|
| nucleic acid name | sequence | NO: |
| miR-21 | UAGCUUAUCAGACUGAUGUUGA | 1 |
| starting hairpin nucleic acid | TCAACATCAGTCTGATAAGCTA | 2 |
| miR-21 binding domain | | |
| elongating hairpin nucleic acid | TAGCTTATCAGACTGATGTTGA | 3 |
| miR-21 domain | | |
| starting hairpin nucleic acid | TCAACATCAGTCTGATAAGCTACCCTGAAAATAGCTTATCAGAC | 4 |
| (5′-end protruding type) | | |
| elongating hairpin nucleic acid | TAGCTTATCAGACTGATGTTGAGTCTGATAAGCTATTTTCAGGG | 5 |
| (3′-end protruding type) | | |
| starting hairpin nucleic acid | TCAACATCAGTCTGATAAGCTAAGTCAGTCTAGCTTATCAGACT | 6 |
| (5′-end protruding type) | | |
| elongating hairpin nucleic acid | TAGCTTATCAGACTGATGTTGAAGTCTGATAAGCTAGACTGACT | 7 |
| (3′-end protruding type) | | |
| starting hairpin nucleic acid | TCAACATCAGTCTGATAAGCTAAGTCAGTCATAGCTTATCAGAC | 15 |
| (5′-end protruding type) | | |
| elongating hairpin nucleic acid | TAGCTTATCAGACTGATGTTGAGTCTGATAAGCTATGACTGACT | 16 |
| (3′-end protruding type) | | |
| starting hairpin nucleic acid | TCAACATCAGTCTGATAAGCTAGACTGACTTAGCTTATCAGACT | 17 |
| (5′-end protruding type) | | |
| elongating hairpin nucleic acid | TAGCTTATCAGACTGATGTTGAAGTCTGATAAGCTAAGTCAGTC | 18 |

(3′-end protruding type)
*In SEQ ID NOs: 2 to 7, and 15 to 18, “T (thymine)” represents “T or U (uracil)”.

A typical HCR reaction is described below, referring to the domains of the hairpin nucleic acids. miR-21 binds to the miR-21 binding domain of the starting hairpin nucleic acid to dissociate the hairpin structure (particularly, the stem structure) of the starting hairpin nucleic acid (step 1 (1) in FIG. 2). Then, the elongating hairpin binding domain of the starting hairpin nucleic acid becomes hybridizable with the starting hairpin binding domain of the elongating hairpin nucleic acid (step 2 (2) in FIG. 2). The 2 domains hybridize with each other to dissociate the hairpin structure (particularly, the stem structure) of the elongating hairpin nucleic acid (steps 2 in FIG. 2). Then, as in step 2, the miR-21 domain of the elongating hairpin nucleic acid becomes hybridizable with the miR-21 binding domain of the starting hairpin nucleic acid (step 3 (3) in FIG. 2). A straight-chain double-stranded nucleic acid (HCR product) having a hybridization chain structure is formed by the chain of such a reaction ((4) and (5) in FIG. 2).

### (3) Domains

The length of each domain comprised in the hairpin nucleic acids is not particularly limited. The length is, for example, 4 bases or more, 5 bases or more, 6 bases or more, 7 bases or more, 8 bases or more, 9 bases or more, 10 bases or more, 11 bases or more, 12 bases or more, 13 bases or more, 14 bases or more, 15 bases or more, 16 bases or more, 17 bases or more, 18 bases or more, 19 bases or more, 20 bases or more, 21 bases or more, or 22 bases or more. Moreover, the length of each domain is, for example, 55 bases or less, 50 bases or less, 40 bases or less, 30 bases or less, 25 bases or less, 24 bases or less, or 23 bases or less.

The base sequence of each domain is not particularly limited as long as the base sequence comprises a base sequence that is hybridizable with a nucleic acid to be hybridized with. For example, each domain may comprise one or a plurality of bases unrelated to the base sequence of a nucleic acid to which the domain binds.

### (3-1) miR-21 Binding Domain and Starting Hairpin Binding Domain

As described above, each of the miR-21 binding domain of the starting hairpin nucleic acid and the starting hairpin binding domain of the elongating hairpin nucleic acid preferably comprises the whole or part of the protruding region, and the whole or part of the subsequent stem region Y. For example, each of the domains may be a consecutive region comprising the whole of the protruding region X, the stem region Y, and the loop region Z, and part of the stem region Y′, or may be a consecutive region comprising part of the protruding region X and the stem region Y. For example, the base sequence hybridizable with miR-21 or with the elongating hairpin binding domain of the starting hairpin nucleic acid, in the stem region comprises 2 bases or more, 3 bases or more, 4 bases or more, 5 bases or more, 6 bases or more, 7 bases or more, 8 bases or more, 9 bases or more, 10 bases or more, 11 bases or more, 12 bases or more, 13 bases or more, 14 bases or more, 15 bases or more, or 16 bases or more.

### (3-2) Elongating Hairpin Binding Domain and miR-21 Domain

As described above, each of the elongating hairpin binding domain of the starting hairpin nucleic acid and the miR-21 domain of the elongating hairpin nucleic acid preferably comprises the whole or part of the stem region Y′. For example, each of the domains may be a consecutive region comprising part of the stem region Y, the whole of the loop region Z, and the whole of the stem region Y′, or may be a consecutive region comprising only part of the stem region Y′. Moreover, when the hairpin nucleic acid comprises a protruding end adjacent to the stem region Y′, the domains may comprise the whole or part of the protruding end. Specifically, for example, the base sequence hybridizable with the miR-21 domain of the elongating hairpin nucleic acid or the miR-21 binding domain of the starting hairpin nucleic acid, in the stem region Y′ comprises 4 bases or more, 5 bases or more, 6 bases or more, 7 bases or more, 8 bases or more, 9 bases or more, 10 bases or more, 11 bases or more, 12 bases or more, 13 bases or more, 14 bases or more, 15 bases or more, or 16 bases or more.

### (4) Hybridization Chain Structure

The hairpin nucleic acid of the present invention forms a straight-chain double-stranded nucleic acid having a hybridization chain structure in the presence of miR-21. The hybridization chain structure can induce nucleic acid immunity.

The specific structure of the hybridization chain structure is not particularly limited as long as the structure is the double-strand structure of a nucleic acid capable of inducing nucleic acid immunity. For example, the hybridization chain structure comprises a pathogen-associated molecular pattern that is recognized by a pattern-recognition receptor existing on a biological membrane or in the cytoplasm.

The pattern-recognition receptor capable of recognizing a nucleic acid existing on the biomembrane includes, for example, Toll-like receptors such as TLR3, TLR7, TLR8, and TLR9. Such receptors exist on the biological membranes of the endosome and lysosome, and recognize a nucleic acid molecule entering a cell.

The pattern-recognition receptor capable of recognizing a nucleic acid existing in the cytoplasm includes, for example, RIG-I-like receptors (for example, RIG-I, MDA5, LGP2, and the like), cGAS, and AIM2.

The pathogen-associated molecular pattern that is recognized by each pattern-recognition receptor is known in the art. For example, TLR3 is known to recognize a double-stranded RNA of 40 base pairs or more, and TLR7 and TLR8 are known to recognize, e.g., a polyuracil, or a double-stranded RNA rich in guanine and uracil. Moreover, TLR9 is known to recognize, e.g., an unmethylated single-stranded DNA comprising a sequence rich in cytosine and guanine, such as 5′-GTCGTT-3′. Further, the RIG-I protein is known to recognize, e.g., a double-stranded RNA comprising a blunt end and having a triphosphate in the 5′-end. In addition, the AIM2 protein is known to recognize, e.g., a double-stranded DNA.

For example, the hybridization chain structure may have a pathogen-associated molecular pattern that is recognized by the pattern-recognition receptor existing in the cytoplasm, or may have a pathogen-associated molecular pattern that is recognized by the pattern-recognition receptor that recognizes a double-stranded nucleic acid (for example, double-stranded DNA and/or double-stranded RNA). For example, the pathogen-associated molecular pattern may be a double-stranded structure that is recognized by cGAS, or may be a double-stranded structure that is recognized by MDA5.

cGAS (cyclic GMP-AMP synthase) is a cyclic GMP-AMP synthase. The exemplary amino acid sequence of cGAS is shown in SEQ ID NO: 14. cGAS bound to a foreign double-stranded nucleic acid (for example, nucleic acid derived from virus) or self abnormal double-stranded nucleic acid (for example, nucleic acid leaked from nucleus in senescent cell or the like) synthesizes 2′-5′-cGAMP, which is a second messenger, from GTP and ATP, to activate a downstream STING (stimulator of interferon genes) pathway through the 2′-5′-cGAMP. The activated STING pathway induces cell apoptosis and the like.

Known examples of nucleic acids that are recognized by cGAS include a long double-stranded DNA that is not included in a chromatin structure, and a short double-stranded DNA having an end comprising guanosine which that is not paired. The immune response pathway caused by cGAS is known to likely be induced, e.g., when a large amount of nucleic acid molecules enter a cell, or when a double-stranded nucleic acid comprising an oxidized DNA molecule enters a cell.

For example, the hybridization chain structure has a certain length. Specifically, the median of the lengths of formed HCR products may be, for example, 100 base pairs or more, 150 base pairs or more, 200 base pairs or more, 250 base pairs or more, 300 base pairs or more, 350 base pairs or more, 400 base pairs or more, 450 base pairs or more, 500 base pairs or more, or 550 base pairs or more. It is preferable to form a constant amount of double-strand structure of 500 base pairs or more. For example, 5% or more, 6% or more, 7% or more, 8% or more, 9% or more, 10% or more, 11% or more, 12% or more, 13% or more, 14% or more, 15% or more, 16% or more, 17% or more, 18% or more, 19% or more, 20% or more, 21% or more, 22% or more, 23% or more, 24% or more, or 25% or more of the HCR products have a double-strand structure of 500 base pairs or more.

### (5) Cell Death-Inducing Composition

Each hairpin nucleic acid comprised in the cell death-inducing composition of the present invention may be composed of DNA and/or RNA nucleotides. Each hairpin nucleic acid may comprise a natural and/or unnatural nucleotide(s) as a composing nucleotide(s). The kinds, numbers, positions, and the like of the comprised natural nucleotides and unnatural nucleotides are not particularly limited.

“Nucleotide” is a compound comprising a phosphate group covalently bound to the sugar moiety of a nucleoside. A “nucleoside” is a combination of a base and a sugar. The nucleobase (also known as a base) moiety of a nucleoside is usually a heterocyclic base moiety. In a nucleoside comprising a pentofuranosyl sugar, a phosphate group can be linked to the 2′, 3′, or 5′ hydroxyl moiety of the sugar. An oligonucleotide is formed by the covalent bonding of nucleosides adjacent to each other, to form a linear polymer oligonucleotide. The phosphate groups inside the oligonucleotide structure are generally considered to form internucleoside bonds of the oligonucleotide.

“Natural nucleotide” herein includes deoxyribonucleotides found in DNA, and ribonucleotides found in RNA. “Deoxyribonucleotide” and “ribonucleotide” herein are sometimes referred to as “DNA nucleotide” and “RNA nucleotide”, respectively.

Similarly, “natural nucleoside” herein includes deoxyribonucleosides found in DNA, and ribonucleosides found in RNA. “Deoxyribonucleoside” and “ribonucleoside” herein are sometimes referred to as “DNA nucleoside” and “RNA nucleoside”, respectively.

“Unnatural nucleotide” refers to any nucleotide other than natural nucleotides, and includes modified nucleotides and nucleotide mimics. Similarly, “non-natural nucleoside” herein refers to any nucleoside other than natural nucleosides, and includes modified nucleosides and nucleoside mimics. “Modified nucleotide” herein means a nucleotide comprising any one or more of a modified sugar moiety, a modified internucleoside bond, and a modified nucleobase. “Modified nucleoside” herein means a nucleoside comprising a modified sugar moiety and/or a modified nucleobase. A nucleic acid comprising an unnatural oligonucleotide may be preferred over a naturally occurring nucleic acid due to, for example, enhanced cellular uptake, enhanced affinity for nucleic acid targets, increased stability in the presence of nucleases, or increased inhibitory activity.

“Modified internucleoside bond” herein refers to an internucleoside bond with a substitution(s) or any change from a naturally occurring internucleoside bond (that is, a phosphodiester bond). The modified internucleoside bond includes internucleoside bonds comprising a phosphorus atom, and internucleoside bonds not comprising a phosphorus atom. Representative phosphorus-containing internucleoside bonds include, but are not limited to, the phosphodiester bond, the phosphorothioate bond, the phosphorodithioate bond, the phosphotriester bond, the methylphosphonate bond, the methylthiophosphonate bond, the boranophosphate bond, and the phosphoramidate bond. The phosphorothioate bond refers to the internucleoside bond formed by replacing a non-bridging oxygen of a phosphodiester bond with a sulfur atom. Methods of preparing phosphorus-containing bonds and non-phosphorus-containing bonds are well known. The modified internucleoside bond preferably has higher nuclease resistance than those of naturally occurring internucleoside bonds.

“Modified nucleobase” or “modified base” herein means any nucleobase other than adenine, cytosine, guanine, thymine, or uracil. “Unmodified nucleobase” or “unmodified base” (natural nucleobase) means adenine (A) and guanine (G), which are purine bases, and thymine (T), cytosine (C), and uracil (U), which are pyrimidine bases. Examples of the modified nucleobase include bases comprising a substituent introduced therein. The type of the base into which the substituent is introduced is not particularly limited. The base may be either a pyrimidine base or a purine base. Further, the position at which the substituent is introduced is not limited. For example, for pyrimidine bases, the position may be the 3′-position, 4′-position, 5′-position, 6′-position, or a combination thereof, and, for purine bases, the position may be the 2′-position, 6′-position, 7′-position, 8′-position, or a combination thereof. Specifically, the modified base includes, but is not limited to, for example, 5-methylcytosine, 5-hydroxymethylcytosine, 5-formylated cytosine, 5-carboxylated cytosine, 5-fluorocytosine, 5-bromocytosine, 5-iodocytosine, or N4-methylcytosine; 2-aminoadenine, N6-methyladenine, 7-deazaadenine, 8-haloadenine, 8-aminoadenine, 8-thioladenine, 8-thioalkyladenine, 8-hydroxyladenine, or 8-bromoadenine; 2-thio-thymine; N2-methylguanine, 6-methylguanine, 7-deazaguanine, 7-methylguanine, 8-haloguanine, 8-aminoguanine, 8-thiolguanine, 8-thioalkylguanine, 8-hydroxylguanine, or 8-bromoguanine; 5-methyluracil, N3-methyluracil, 6-methyluracil, 5-fluorouracil, 5-bromouracil, 5-iodouracil, 5-hydroxyuracil, pseudouracil, or methylpseudouracil; hypoxanthine or xanthine; and derivatives thereof.

As used herein, “modified sugar” refers to a sugar with a substitution(s) and/or any change from a natural sugar moiety (that is, the sugar moiety found in DNA (2′-H) or RNA (2′-OH)). In some cases, the nucleic acid herein may comprise one or more modified nucleosides comprising a modified sugar. The sugar modification may enhance the stability against nucleases, increase the binding affinity, or impart some other beneficial biological properties to the nucleic acid. For example, the nucleoside may comprise a chemically modified ribofuranose ring moiety. Examples of the chemically modified ribofuranose ring include, but are not limited to, addition of a substituent (including 5′ and 2′ substituents), formation of a bicyclic nucleic acid (bridged nucleic acid, BNA) by bridging between non-geminal ring atoms, substitution of the ribosyl ring oxygen atom with S, N(R), or C(R₁)(R₂) (wherein R, R₁, and R₂ each independently represent H, C₁-C₁₂ alkyl, or a protecting group), and combinations thereof.

Herein, examples of the nucleoside having a modified sugar moiety include, but are not limited to, nucleosides comprising 5′-vinyl, 5′-methyl, 4′-S, 2′-F (2′-fluoro group), 2′-OCH₃ (2′-OMe group or 2′-O-methyl group), and 2′-O(CH₂)₂OCH₃ substituents. Further, the substituent at 2′-position may be selected from allyl, amino, azide, thio, —O-allyl, —O—C₁-C₁₀ alkyl, —OCF₃, —O(CH₂)₂SCH₃, —O(CH₂)₂—O—N(Rₘ)(Rₙ), and —O—CH₂—C(═O)—N(Rₘ)(Rₙ), wherein Rₘ and Rₙ each independently represent H, or substituted or unsubstituted C₁-C₁₀ alkyl. “2′-modified sugar” herein means a furanosyl sugar modified at the 2′-position.

As used herein, “bicyclic nucleoside” refers to a modified nucleoside comprising a bicyclic sugar moiety. A nucleic acid comprising a bicyclic sugar moiety is generally referred to as a bridged nucleic acid (BNA). Herein, a nucleoside comprising a bicyclic sugar moiety may be referred to as a “bridged nucleoside”.

The bicyclic sugar may be a sugar in which the carbon atom at the 2′-position and the carbon atom at the 4′-position are bridged through 2 or more atoms. Examples of bicyclic sugars are known to those skilled in the art. One subgroup of bicyclic sugar-comprising nucleic acids (BNAs) has a carbon atom at the 2′-position and a carbon atom at the 4′-position bridged through 4′-(CH₂)ₚ—O-2′, 4′-(CH₂)ₚ—CH₂-2′, 4′-(CH₂)ₚ—S-2′, 4′-(CH₂)ₚ—OCO-2′, or 4′-(CH₂) n-N(R₃)—O—(CH₂)ₘ-2′ [wherein p, m, and n each independently represent an integer of 1 to 4, an integer of 0 to 2, and an integer of 1 to 3, respectively; and R₃ represents a hydrogen atom, an alkyl group, an alkenyl group, a cycloalkyl group, an aryl group, an aralkyl group, an acyl group, a sulfonyl group, and a unit substituent (for example, a fluorescent or chemiluminescent labeling molecule, a functional group having nucleic acid cleavage activity, or a cellular or nuclear localization signal peptide)]. In the OR₂ substituent on the carbon atom at the 3′-position and the OR₁ substituent on the carbon atom at the 5′-position of BNA, R₁ and R₂ are typically hydrogen atoms, but may also be any other substituents. For example, the substituents may be the same or different from each other. Specifically, R₁ and R₂ each independently include, for example, a protecting group for a hydroxyl group for nucleic acid synthesis, an alkyl group, an alkenyl group, a cycloalkyl group, an aryl group, an aralkyl group, an acyl group, a sulfonyl group, a silyl group, a phosphate group, a phosphate group protected by a protecting group for nucleic acid synthesis, or —P(R₄)R₅ [wherein R₄ and R₅, which may be the same or different from each other, each represent a hydroxyl group, a hydroxyl group protected by a protecting group for nucleic acid synthesis, a mercapto group, a mercapto group protected by a protecting group for nucleic acid synthesis, an amino group, an alkoxy group having 1 to 5 carbon atoms, an alkylthio group having 1 to 5 carbon atoms, a cyanoalkoxy group having 1 to 6 carbon atoms, or an amino group substituted with an alkyl group having 1 to 5 carbon atoms]. Non-limiting examples of such BNAs include methyleneoxy(4′-CH₂—O-2′) BNA (also known as LNA (Locked Nucleic Acid (registered trademark)) or 2′,4′-BNA), such as α-L-methyleneoxy(4′-CH₂—O-2′) BNA or β-D-methyleneoxy(4′-CH₂—O-2′) BNA; ethyleneoxy(4′-(CH₂)₂—O-2′) BNA (also known as ENA); β-D-thio(4′-CH₂—S-2′) BNA; aminooxy(4′-CH₂—O—N(R₃)-2′) BNA; oxyamino(4′-CH₂—N(R₃)—O-2′) BNA (also known as 2′,4′-BNA^{coc}); 2′,4′-BNA^{coc}; 3′-amino-2′,4′-BNA; 5′-methyl BNA; (4′-CH(CH₃)—O-2′) BNA (also known as cEt BNA);(4′-CH(CH₂OCH₃)—O-2′) BNA (also known as cMOE BNA); amide BNA (4′-C(O)—N(R)-2′) BNA (R=H, Me) (also known as AmNA); 2′-O,4′-C-spirocyclopropylene bridged nucleic acid (also known as scpBNA); and other BNAs known to those skilled in the art.

Methods of preparing modified sugars are well known to those skilled in the art. In a nucleotide comprising a modified sugar moiety, the nucleobase moiety (a natural moiety, a modified moiety, or a combination thereof) may be maintained for hybridization with an appropriate nucleic acid target.

“Nucleoside mimic” herein includes structures used to replace sugars, sugars and bases, or sugars, bases, and bonds, at one or more positions in oligomeric compounds. The nucleoside mimic includes, for example, morpholino, cyclohexenyl, cyclohexyl, tetrahydropyranyl, bicyclic, or tricyclic sugar mimics, such as nucleoside mimics comprising a non-furanose sugar unit. “Nucleotide mimic” includes structures used to replace nucleosides and bonds at one or more positions in oligomeric compounds. The nucleotide mimic includes, for example, peptide nucleic acids or morpholino nucleic acids (morpholinos formed by binding through —N(H)—C(═O)—O— or other non-phosphodiester bonds). Peptide nucleic acid (PNA) is a nucleotide mimic having a backbone formed by binding of N-(2-aminoethyl)glycine instead of sugars through amide bonds. “Mimic” refers to a group that substitutes a sugar, a nucleobase, and/or an internucleoside bond. In general, mimics are used in place of sugars or a combination of sugars and internucleoside bonds, and the nucleobases are maintained for hybridization to a selected target.

The specific mode of the modification is not particularly limited. For example, nucleotides in the same nucleic acid molecule may be differently modified independently. Further, for example, a plurality of types of modifications may be made in one nucleotide. For example, a modified internucleoside bond (for example, a phosphorothioate bond) and a modified sugar (for example, a 2′-O-methyl-modified sugar or a bicyclic sugar) may be comprised in a nucleotide for the purpose of, for example, imparting resistance against enzymatic cleavage. Further, for example, a modified nucleobase (for example, 5-methylcytosine) and a modified sugar (for example, a 2′-O-methyl-modified sugar or a bicyclic sugar) may be comprised. Further, for example, one or more of the starting hairpin nucleic acid and the elongating hairpin nucleic acid contain neither a natural nucleoside nor an unmodified nucleotide over the entire length. Further, for example, the number of non-natural nucleosides and modified nucleotides comprised in one or more of the starting hairpin nucleic acid and the elongating hairpin nucleic acid is half or less over the entire length. Further, for example, both the starting hairpin nucleic acid and the elongating hairpin nucleic acid comprise neither a natural nucleoside nor an unmodified nucleotide. Alternatively, for example, in one or more of the starting hairpin nucleic acid and the elongating hairpin nucleic acid, all nucleotides comprised in the protruding region X may be a non-natural nucleoside(s) and/or a modified nucleotide(s), and the internucleoside bond linking the protruding region X to the stem region Y may be a modified internucleoside bond.

In the selection of the modification, those skilled in the art may determine a preferred embodiment with reference to the description of literature related to a nucleic acid medicine (for example, WO 2007/143315). Moreover, the objective of the modification is not particularly limited. For example, the modification may be carried out for stabilizing and detecting the hairpin nucleic acids and the HCR product, and for the performance of the pharmacology functions of the hairpin nucleic acids and the HCR product.

The starting hairpin nucleic acid and the elongating hairpin nucleic acid comprised in the cell death-inducing composition may comprise a plurality of kinds of hairpin nucleic acids.

The nucleic acid molecules herein may be prepared by any method. For example, the nucleic acid molecules may be fully or partially prepared by a chemical synthesis method (for example, using an automatic synthesizer) or an enzymatic process (for example, reaction by a polymerase, a ligase, or a restriction enzyme, but the process is not limited thereto).

The cell death-inducing composition of the present invention is intended to induce cell death. Accordingly, the cell death-inducing composition may comprise one or more active ingredients capable of inducing cell death as active ingredients, in addition to the hairpin nucleic acids described above. Specific examples of the active ingredients include compounds, drugs based on the promotion or suppression of gene expression, and drugs based on the promotion or inhibition of functions at the protein levels, that induce cell death.

It is not necessary that the cell death-inducing composition of the present invention induces cell death only in a cell comprising an HCR product. For example, when the hybridization chain structure is a structure that is recognized by cGAS, 2′-5′-cGAMP, which is a second messenger synthesized by cGAS, may migrate to adjacent cells and the like through gap junctions to also induce cell death in those cells. For example, the cell death of a cell comprising an HCR product may result in the induction of an immune response. The immune response may result in the promotion of secondary cell death (for example, immunogenic cell death) in a cell that does not comprise any HCR product but has a similar abnormality.

Thus, the cell death-inducing composition herein can induce cell death in cells expressing miR-21 and also expressing a pattern-recognition receptor such as cGAS, as well as in other cells expressing miR-21.

### 2. Pharmaceutical Composition

### 2-1. Summary

The second aspect of the present invention is a pharmaceutical composition. The pharmaceutical composition of the present invention comprises the cell death-inducing composition described in the first aspect as an active ingredient, and induces HCR reaction in a site of interest comprising miR-21. By using the pharmaceutical composition of the present invention, cell death can be induced specifically for miR-21-expressing cells in the site of interest.

### 2-2. Components

The components of the pharmaceutical composition of the present aspect are described below. The pharmaceutical composition of the present invention comprises a carrier as an optionally selectable component, in addition to an active ingredient as an essential component. Each component is specifically described below.

### (1) Active Ingredient

The pharmaceutical composition of the present invention comprises an effective amount of the cell death-inducing composition described in the first aspect, as an essential active ingredient. Since the components of the cell death-inducing composition are described in detail in the first aspect, specific descriptions thereof are omitted. One or more additional active ingredients may be comprised depending on the desired effect achieved by the pharmaceutical composition.

“Effective amount” refers to an amount conferring a subject to which the composition is applied, little or no harmful side effect, and required for performing the function of the cell death-inducing composition as the active ingredient. The effective amount may be changed by various conditions such as information on the subject, the route of administration, and the number of doses. The final decision is made by the discretion of a person who performs the administration, including a doctor, a veterinarian, or a pharmacist.

For example, when the hybridization chain structure comprises a structure that is recognized by cGAS, the effective amount may be determined so that the hybridization chain structure in an amount sufficient for activating cGAS is formed in a cell. Specifically, for example, the effective amount may be set so that the amount of a nucleic acid in a target cell is 0.01 nM to 20 nM, 0.05 nM to 15 nM, 0.08 nM to 12 nM, 0.09 nM to 11 nM, 0.1 nM to 10 nM, 0.11 nM to 10 nM, 0.2 nM to 10 nM, 0.5 nM to 10 nM, 0.8 nM to 10 nM, 0.9 nM to 10 nM, 1 nM to 10 nM, 2 nM to 10 nM, 5 nM to 10 nM, 0.1 nM to 9 nM, 0.1 nM to 8 nM, 0.1 nM to 6 nM, 0.1 nM to 5 nM, 0.1 nM to 3 nM, 0.1 nM to 2 nM, 0.1 nM to 1 nM, 0.2 nM to 0.9 nM, or 0.3 nM to 0.8 nM, without particular limitation. Further, for example, the concentration of each hairpin nucleic acid may be 1.5 or more times, 2 or more times, 2.5 or more times, 3 or more times, 3.5 or more times, 4 or more times, 4.5 or more times, 5 or more times, 5.5 or more times, 6 or more times, 6.5 or more times, 7 or more times, 7.5 or more times, 8 or more times, 8.5 or more times, 9 or more times, 9.5 or more times, or 10 or more times higher than the concentration of miR-21 in the cell. The concentrations of the hairpin nucleic acids may be the same or different.

“Subject” herein refers to a subject to which the cell death-inducing composition and the pharmaceutical composition of the present invention are applied. The subject may include an organ, a tissue, and a cell, as well as an individual. The subject may be any animal, including a human, when being an individual. For example, the animal includes various domestic animals, poultry, companion animals, and experimental animals, as well as humans. Without limitation, the subject may be an individual having abnormal expression of a protein, or an abnormal cell, or may be an individual requiring treatment or prevention of a disease.

“Information on subject” herein refers to various information of an individual on a living body to which the application is performed. When the subject is a human, the information includes, for example, age, body weight, gender, dietary habits, health condition, the degree of progression and severity of a disease, drug sensitivity, and the presence or absence of a concomitant drug.

The pharmaceutical composition of the present invention may be the so-called combination formulation, which comprises another active ingredient to an extent at which the pharmacological effect of the hairpin nucleic acids as active ingredients is not lost. The “other active ingredient” herein includes, for example, drugs that target miR-21-expressing cells to induce their cell death by an action mechanism different from that of the cell death-inducing composition of the first aspect. Further, the other active ingredient may be a drug having a pharmacological action different from that of the cell death-inducing composition of the first aspect. Such a drug includes, for example, antibiotics.

### (2) Carrier

The pharmaceutical composition of the present invention may comprise a pharmaceutically acceptable carrier. “Pharmaceutically acceptable carrier” refers to an additive that is commonly used in the technical field of pharmaceutical preparation. For example, solvents, bases, emulsifiers, suspending agents, surfactants, pH adjusters, stabilizing agents, flavoring agents, excipients, vehicles, antiseptic agents, binders, diluents, isotonizing agents, sedatives, buffer agents, coating agents, lubricants, coloring agents, thickeners, dissolution auxiliaries, and other additives are included.

The solvents include any solvents, such as water or another pharmaceutically acceptable aqueous solution, or a pharmaceutically acceptable organic solvent (for example, vegetable oil or the like). The aqueous solution includes, for example, physiological saline, isocratic liquids comprising glucose and other auxiliary agents, phosphate buffers, and sodium acetate buffers. The auxiliary agents include, for example, nonionic surfactants having low concentration, and polyoxyethylene sorbitan fatty acid esters, as well as D-sorbitol, D-mannose, D-mannitol, and sodium chloride.

The carrier is used to avoid or suppress in vivo decomposition of the cell death-inducing composition, which is an active ingredient, caused by an enzyme and/or the like, as well as to facilitate formulation and an administration method, and to maintain dosage form and drug efficacy, and may be used as appropriate, as needed.

### (3) Dosage Form

The dosage form of the pharmaceutical composition of the present invention is not particularly limited as long as being a form in which the cell death-inducing composition according to the first aspect, which is an active ingredient, can be delivered to a target site without being inactivated by decomposition and/or the like, and the pharmacological effect of the active ingredient can be produced in vivo.

The specific dosage form depends on the administration form and/or a prescription condition. The administration form of the pharmaceutical composition of the present invention may be classified roughly into oral administration and parenteral administration. When the administration method is parenteral administration, a preferred dosage form is a liquid that can be directly administered to a target site or systemically administered through the circulatory system. Examples of the liquid include injectable agents. An injectable agent may be formulated in combination with the excipient, an elixir, an emulsifier, a suspension, a surfactant, a stabilizer, a pH regulator, or the like, as appropriate, and mixing the resultant in a unit dosage form required for performing commonly approved medicine manufacture. In addition, the dosage form may be an ointment, a plaster, a cataplasm, a transdermal agent, a lotion, an inhalant, an aerosol, an ophthalmic solution, or a suppository.

Both the specific shape and size of each of the dosage forms described above may be within the ranges of the dosage form known in the art, and are not particularly limited. With regard to a method of producing the pharmaceutical composition of the present invention, formulation may be performed according to a common method in the art.

The cell death-inducing composition of the present invention is excellent in solubility in water, Japanese Pharmacopeia second fluid for dissolution test, or Japanese Pharmacopeia second fluid for disintegration test, and has properties excellent for a pharmaceutical product, for example, is excellent in pharmacokinetics (for example, drug half-life in blood, intracerebral transferability, metabolic stability, and CYP inhibition), has low toxicity (for example, superiority as a medicine in view of acute toxicity, chronic toxicity, genetic toxicity, reproductive toxicity, cardiotoxicity, drug interaction, carcinogenicity, phototoxicity, or the like), and also has fewer side effects (for example, suppression of sedation, or avoidance of laminar necrosis).

### (4) Administration Style and Dose

Herein, a preferred style of administration of the pharmaceutical composition of the present invention is not specifically limited. For example, the style may be oral administration or parenteral administration. The parenteral administration is usually used.

Specific examples of the parenteral administration include intramuscular administration, intravenous administration, intraarterial administration, intraperitoneal administration, subcutaneous administration (including implant-type continuous subcutaneous administration), intradermal administration, trachea/bronchus administration, rectal administration, administration via blood transfusion, intratumoral administration, peritumoral administration (for example, intradermal administration or subcutaneous administration at peritumoral site), intracerebroventricular administration, intrathecal administration, nasal administration, and intramuscular administration. The administration may be carried out by intramuscular injection administration, intravenous infusion, or implanted continuous subcutaneous administration. The subcutaneous administration includes, for example, self-injection by the patient.

Even repetitive administration of the pharmaceutical composition of the present invention additively produces a suppressive effect in a cell. In the case of such repetitive administration, efficacy may be improved at an administration interval to a certain degree (for example, half a day or more).

### (5) Target Diseases

The diseases to which the pharmaceutical composition of the present invention is applied are not particularly limited as long as the diseases show high expression of miR-21. The diseases are typically malignant tumors (cancers) and inflammatory diseases.

The malignant tumors (cancers) herein include, for example, breast cancer, colorectal cancer, pancreatic cancer, lung cancer, prostate cancer, liver cancer, gastric cancer, brain tumor, thyroid cancer, oral cancer, acute myeloid leukemia, chronic lymphocytic leukemia, glioblastoma, esophageal cancer, hepatocellular carcinoma, glioma, cervical cancer, bladder cancer, laryngeal cancer, pancreatic ductal adenocarcinoma, head and neck squamous cell carcinoma, oral squamous cell carcinoma, ovarian cancer, cholangiocarcinoma, renal cell carcinoma, skin cancer, endometrial cancer, bile duct cancer, kidney cancer, malignant melanoma, and rectal cancer. The cancers herein include their pathologies after metastasis.

For example, when a target disease is a cancer, an anticancer agent comprising the cell death-inducing composition of the present invention as an active ingredient may be made by components in accordance with those of the pharmaceutical composition of the present aspect.

The anticancer agent of the present invention may optionally comprise another additional anticancer agent.

For example, when an inflammatory disease is a target disease, an anti-inflammatory agent comprising the cell death-inducing composition of the present invention as an active ingredient may be made by components in accordance with those of the pharmaceutical composition of the present aspect.

“Inflammatory disease” herein refers to a disease characterized by high-level inflammation or degeneration in a tissue. In particular, the inflammatory disease herein includes both chronic inflammatory diseases and acute inflammatory diseases. Specifically, the inflammatory disease includes, for example, atopic dermatitis, contact dermatitis, allergic contact dermatitis, and arthritis.

### EXAMPLES

### Example 1: Evaluation of HCR Efficiency in Cell-Free System

### Purpose

To examine the relationship between HCR efficiency and the structures of hairpin nucleic acids in the experimental system of a cell-free system.

### Method

### 1. Design of Hairpin Nucleic Acids

Three sets of hairpin nucleic acids in which miR-21 (SEQ ID NO: 1) was a target RNA were designed using online software NUPACK (http://www.nupack.org/). The base sequence of each hairpin nucleic acid is shown in Table 2.

TABLE 2
Sequences of hairpin nucleic acids used in Example 1

| nucleic | | |
|---|---|---|
| acid | | SEQ ID |
| name | sequence (5′→3′) | NO: |
| HP-o(1) | T^{∧}C^{∧}A^{∧}A^{∧}C^{∧}A^{∧}T^{∧}C^{∧}AGTCTGATAAGCTAGGGACTTTCCTAGCTTATCAGACT | 8 |
| HP-o(2) | TAGCTTATCAGACTGATGTTGAAGTCTGATAAGCTA^{∧}G^{∧}G^{∧}A^{∧}A^{∧}A^{∧}G^{∧}T^{∧}C^{∧}C^{∧}C | 9 |
| HP-a(1) | T^{∧}C^{∧}A^{∧}A^{∧}C^{∧}A^{∧}T^{∧}C^{∧}A^{∧}GTCTGATAAGCTACCCTGAAAATAGCTTATCAGAC | 10 |
| HP-a(2) | TAGCTTATCAGACTGATGTTGAGTCTGATAAGCTA^{∧}T^{∧}T^{∧}T^{∧}T^{∧}C^{∧}A^{∧}G^{∧}G^{∧}G | 11 |
| HP-b(1) | T^{∧}C^{∧}A^{∧}A^{∧}C^{∧}A^{∧}T^{∧}C^{∧}AGTCTGATAAGCTAAGTCAGTCTAGCTTATCAGACT | 12 |
| HP-b(2) | TAGCTTATCAGACTGATGTTGAAGTCTGATAAGCTA^{∧}G^{∧}A^{∧}C^{∧}T^{∧}G^{∧}A^{∧}C^{∧}T | 13 |

^{∧}: phosphorothioate; underline: protruding region X; boldface: stem region Y

### 2. Evaluation of HCR Efficiency

For each set of the hairpin nucleic acids, miR-21 was added, at a final concentration of 50 nM, to 100 L of TE buffering solution (comprising 500 mM NaCl and 1.25 mM MgCl₂) in which each of a starting hairpin nucleic acid (HP (1)) and an elongating hairpin nucleic acid (HP (2)) was comprised at a concentration of 500 nM, and the resultant liquid mixture was incubated at 37° C. for 2 hours. A liquid mixture to which miR-21 was not added was used as a control. Analysis by native PAGE (non-denaturing PAGE) was carried out using 5% polyacrylamide gel, and the gel was stained for 15 minutes using SYBR™ Gold (Invitrogen), followed by visualization using Gel Doc EZ Imager (BioRad).

### Results

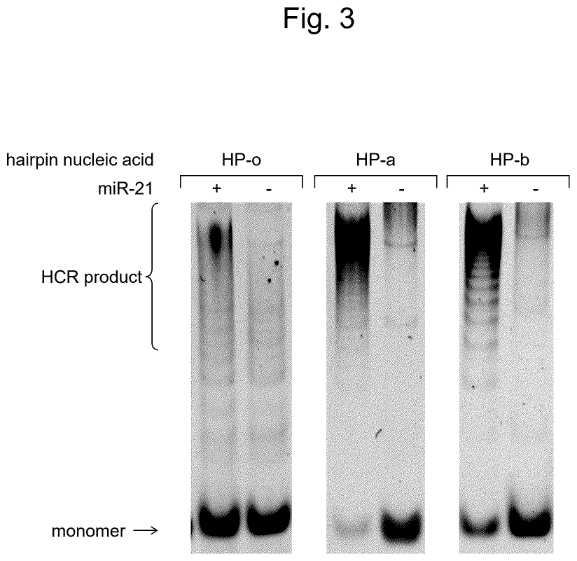

The results are indicated in FIG. 3.

HCR was induced in any of the three kinds of evaluated sets of the hairpin nucleic acids. In all the sets, a number of monomeric hairpin nucleic acid molecules existed when no miR-21 was added, and the monomers were prominently decreased by adding miR-21, resulting in the appearance of HCR products with high molecular weights. The set of the hairpin nucleic acids HP-a and HP-b caused a prominent decrease in the amount of monomers under the conditions where miR-21 was present, resulting in the formation of more HCR products compared to the set of the hairpin nucleic acids for comparison (HP-o). It was thus suggested that the set of the hairpin nucleic acids HP-a and HP-b shows a prominently high efficiency of formation of HCR products.

### Example 2: Evaluation of HCR Efficiency in Cell-Free System (2)

### Purpose

To examine the relationship between HCR efficiency and the structures of hairpin nucleic acids in the experimental system of a cell-free system.

### Method

As in Example 1, hairpin nucleic acids having the sequences shown in Table 3 below were designed.

TABLE 3
Sequences of hairpin nucleic acids used in Example 2

| nucleic | | |
|---|---|---|
| acid | | SEQ ID |
| name | sequence (5′→3′) | NO: |
| HP-c(1) | T^{∧}C^{∧}A^{∧}A^{∧}C^{∧}A^{∧}T^{∧}C^{∧}A^{∧}GTCTGATAAGCTAAGTCAGTCATAGCTTATCAGAC | 19 |
| HP-c(2) | TAGCTTATCAGACTGATGTTGAGTCTGATAAGCTA^{∧}T^{∧}G^{∧}A^{∧}C^{∧}T^{∧}G^{∧}A^{∧}C^{∧}T | 20 |
| HP-d(1) | T^{∧}C^{∧}A^{∧}A^{∧}C^{∧}A^{∧}T^{∧}C^{∧}AGTCTGATAAGCTAGACTGACTTAGCTTATCAGACT | 21 |
| HP-d(2) | TAGCTTATCAGACTGATGTTGAAGTCTGATAAGCTA^{∧}A^{∧}G^{∧}T^{∧}C^{∧}A^{∧}G^{∧}T^{∧}C | 22 |

^{∧}: phosphorothioate; underline: protruding region X; boldface: stem region Y

The HCR efficiency was evaluated as follows.

For each set of the hairpin nucleic acids, miR-21 was added, at a final concentration of 0.1 μM, to a TE buffering solution in which each of HP (1) and HP (2) was comprised at a concentration of 1 M, and the resultant liquid mixture was left at room temperature. A liquid mixture to which miR-21 was not added was used as a control. The reaction solution was analyzed by 1% agarose gel electrophoresis after incubation. For staining the gel, GelRed (Biotium) was used.

### Results

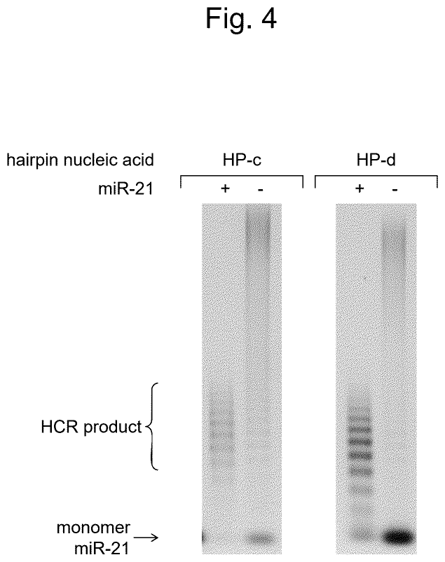

The results are indicated in FIG. 4.

As in Example 1, for both sets of 2 kinds of hairpin nucleic acids evaluated in the present Example, HCR was specifically induced in the presence of miR-21.

### Example 3: Evaluation of Efficiency of Promotion of Cell Death by HCR

### Purpose

Cell death was confirmed to be promoted by HCR in human cells, and the efficiency thereof was examined.

### Method

In a 96-well multi-well plate, 1.0×10⁵ HeLa cells were seeded and cultured in 200 L of DMEM solution containing 10% FBS and 0.5% penicillin-streptomycin until reaching about 90% confluency.

For the sets of 2 kinds of hairpin nucleic acids shown in Table 3, lipofectamine LTX (Thermo Fisher Scientific) and 50 L of OPTI-MEM were added to a mixed solution of 50 L of OPTI-MEM (Thermo Fisher Scientific) and 1 L of Lipofectamine Plus Reagent (Thermo Fisher Scientific), comprising HP (1) and HP (2) at a concentration of 0 g/L or 0.5 g/L, and the resultant was left at room temperature.

Thereafter, the reaction solution was placed in a dish, and, 2 hours later, the medium was replaced with 200 L of DMEM solution containing 10% FBS and 0.5% penicillin-streptomycin. Twenty-four hours later, the medium was replaced with 100 L of DMEM solution containing 9% FBS, 0.45% penicillin-streptomycin, and 10% PrestoBlue (Invitrogen).

Cell death was detected by measuring the fluorescence of PrestoBlue. A relative cell survival rate was calculated as a relative value to a cell survival rate, which was taken as 100%, observed when hairpin nucleic acids having scrambled sequences were introduced. The experiment was repeated 3 times.

### Results

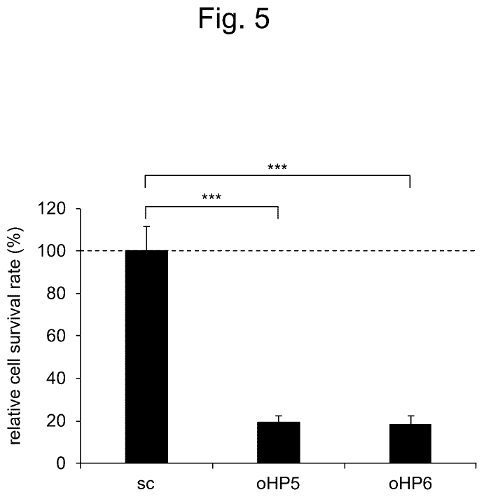

The results are indicated in FIG. 5.

The introduction of the hairpin nucleic acids to the HeLa cells expressing miR-21 resulted in a prominent decrease in the cell survival rate, indicating that the introduction of the hairpin nucleic acids can promote cell death through HCR in a manner specific to cells in which a target RNA exists. Further, the efficiency of inducing cell death was prominently high.

All publications, patents, and patent application cited in the present specification are incorporated herein by reference in their entirety.

1. A cell death-inducing composition comprising a set of hairpin nucleic acids consisting of a starting hairpin nucleic acid comprising a structure represented by general formula (I) and an elongating hairpin nucleic acid comprising a structure represented by general formula (II), wherein the cell death is induced specifically in a cell expressing miR-21 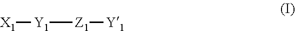
wherein,
X₁ is a protruding region consisting of a sequence of 4 to 20 bases;
Y₁ and Y′₁ are stem regions consisting of sequences of 10 to 20 bases capable of hybridizing with each other intramolecularly;
Z₁ is a loop region consisting of a sequence of 7 to 12 bases without a CG sequence and a GGG sequence;
a whole X₁ and a whole or part of Y₁ constitute a miR-21 binding domain capable of hybridizing with a whole or part of the base sequence of miR-21; and
a whole or part of Y′₁ constitutes an elongating hairpin binding domain capable of hybridizing with a whole or part of miR-21 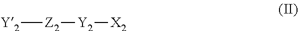
wherein,
X₂ is a protruding region consisting of a sequence of 4 to 20 bases;
Y₂ and Y′₂ are stem regions consisting of sequences of 10 to 20 bases capable of hybridizing with each other intramolecularly;
Z₂ is a loop region consisting of a sequence of 7 to 12 bases;
a whole or part of Y′₂ constitutes a miR-21 domain; and
a whole Y₂ and a whole or part of X₂ constitute a starting hairpin binding domain miR-21 domain capable of hybridizing with a whole or part of the elongating hairpin binding domain.

2. The cell death-inducing composition according to claim 1, wherein the left side in the formula (I) and the formula (II) is the 5′ terminus.

3. The cell death-inducing composition according to claim 1, wherein the miR-21 binding domain comprises the base sequence shown in SEQ ID NO: 2, and the miR-21 domain comprises the base sequence shown in SEQ ID NO: 3.

4. The cell death-inducing composition according to claim 1, wherein the starting hairpin nucleic acid forms a single-stranded structure in the presence of miR-21 under mammalian physiological conditions.

5. The cell death-inducing composition according to claim 1, wherein the GC content of the Z₁ is 40% or more and 60% or less.

6. The cell death-inducing composition according to claim 1, wherein the content of purine bases in the Z₁ is 40% or more and 60% or less.

7. The cell death-inducing composition according to claim 1, wherein the hairpin nucleic acid is composed of DNA and/or RNA nucleotides.

8. The cell death-inducing composition according to claim 1, comprising one or more modified nucleotides and/or unnatural nucleotides.

9. The cell death-inducing composition according to claim 8, wherein the modified nucleotide or the unnatural nucleotide comprises a base into which a substituent is introduced.

10. The cell death-inducing composition according to claim 8, wherein the modified nucleotide or the unnatural nucleotide comprises a modified internucleoside bond.

11. A pharmaceutical composition comprising the cell death-inducing composition according to claim 1.

12. A method for preventing or treating cancer or an inflammatory disease, comprising administering a composition of claim 1 to subject in needed thereof.

13. The method according to claim 12, wherein the cancer is one or more cancers selected from the group consisting of breast cancer, colorectal cancer, pancreatic cancer, lung cancer, prostate cancer, liver cancer, gastric cancer, brain tumor, thyroid cancer, oral cancer, acute myeloid leukemia, chronic lymphocytic leukemia, and glioblastoma.

14. (canceled)

15. The method of claim 12, wherein the cancer comprises cells expressing miR-21.


SEQUENCE LISTING (22 sequences)

SEQ ID NO: 1 - RNA, 22 nt, Homo sapiens
  source 1..22: mol_type = transcribed RNA
tagcttatcagactgatgttga

SEQ ID NO: 2 - DNA, 22 bp, synthetic construct
  source 1..22: mol_type = other DNA
  misc_difference 1: note = t is uracil or thymine
  misc_difference 7: note = t is uracil or thymine
  misc_difference 11: note = t is uracil or thymine
  misc_difference 13: note = t is uracil or thymine
  misc_difference 16: note = t is uracil or thymine
  misc_difference 21: note = t is uracil or thymine
tcaacatcagtctgataagcta

SEQ ID NO: 3 - DNA, 22 bp, synthetic construct
  source 1..22: mol_type = other DNA
  misc_difference 1: note = t is uracil or thymine
  misc_difference 5: note = t is uracil or thymine
  misc_difference 6: note = t is uracil or thymine
  misc_difference 8: note = t is uracil or thymine
  misc_difference 14: note = t is uracil or thymine
  misc_difference 17: note = t is uracil or thymine
  misc_difference 19: note = t is uracil or thymine
  misc_difference 20: note = t is uracil or thymine
tagcttatcagactgatgttga

SEQ ID NO: 4 - DNA, 44 bp, synthetic construct
  source 1..44: mol_type = other DNA
  misc_difference 1: note = t is uracil or thymine
  misc_difference 7: note = t is uracil or thymine
  misc_difference 11: note = t is uracil or thymine
  misc_difference 13: note = t is uracil or thymine
  misc_difference 16: note = t is uracil or thymine
  misc_difference 21: note = t is uracil or thymine
  misc_difference 26: note = t is uracil or thymine
  misc_difference 32: note = t is uracil or thymine
  misc_difference 36..37: note = t is uracil or thymine
  misc_difference 39: note = t is uracil or thymine
tcaacatcagtctgataagctaccctgaaaatagcttatcagac

SEQ ID NO: 5 - DNA, 44 bp, synthetic construct
  source 1..44: mol_type = other DNA
  misc_difference 1: note = t is uracil or thymine
  misc_difference 5..6: note = t is uracil or thymine
  misc_difference 8: note = t is uracil or thymine
  misc_difference 14: note = t is uracil or thymine
  misc_difference 17: note = t is uracil or thymine
  misc_difference 19..20: note = t is uracil or thymine
  misc_difference 24: note = t is uracil or thymine
  misc_difference 26: note = t is uracil or thymine
  misc_difference 29: note = t is uracil or thymine
  misc_difference 34: note = t is uracil or thymine
  misc_difference 36..39: note = t is uracil or thymine
tagcttatcagactgatgttgagtctgataagctattttcaggg

SEQ ID NO: 6 - DNA, 44 bp, synthetic construct
  source 1..44: mol_type = other DNA
  misc_difference 1: note = t is uracil or thymine
  misc_difference 7: note = t is uracil or thymine
  misc_difference 11: note = t is uracil or thymine
  misc_difference 13: note = t is uracil or thymine
  misc_difference 16: note = t is uracil or thymine
  misc_difference 21: note = t is uracil or thymine
  misc_difference 25: note = t is uracil or thymine
  misc_difference 29: note = t is uracil or thymine
  misc_difference 31: note = t is uracil or thymine
  misc_difference 35..36: note = t is uracil or thymine
  misc_difference 38: note = t is uracil or thymine
  misc_difference 44: note = t is uracil or thymine
tcaacatcagtctgataagctaagtcagtctagcttatcagact

SEQ ID NO: 7 - DNA, 44 bp, synthetic construct
  source 1..44: mol_type = other DNA
  misc_difference 1: note = t is uracil or thymine
  misc_difference 5..6: note = t is uracil or thymine
  misc_difference 8: note = t is uracil or thymine
  misc_difference 14: note = t is uracil or thymine
  misc_difference 17: note = t is uracil or thymine
  misc_difference 19..20: note = t is uracil or thymine
  misc_difference 25: note = t is uracil or thymine
  misc_difference 27: note = t is uracil or thymine
  misc_difference 30: note = t is uracil or thymine
  misc_difference 35: note = t is uracil or thymine
  misc_difference 40: note = t is uracil or thymine
  misc_difference 44: note = t is uracil or thymine
tagcttatcagactgatgttgaagtctgataagctagactgact

SEQ ID NO: 8 - DNA, 46 bp, synthetic construct
  modified_base 1..9: mod_base = OTHER; note = internucleoside bonds are phosphorothioate bonds
  source 1..46: mol_type = other DNA
tcaacatcagtctgataagctagggactttcctagcttatcagact

SEQ ID NO: 9 - DNA, 46 bp, synthetic construct
  modified_base 36..46: mod_base = OTHER; note = internucleoside bonds are phosphorothioate bonds
  source 1..46: mol_type = other DNA
tagcttatcagactgatgttgaagtctgataagctaggaaagtccc

SEQ ID NO: 10 - DNA, 44 bp, synthetic construct; identical to SEQ ID NO: 4
  modified_base 1..10: mod_base = OTHER; note = internucleoside bonds are phosphorothioate bonds
  source 1..44: mol_type = other DNA

SEQ ID NO: 11 - DNA, 44 bp, synthetic construct; identical to SEQ ID NO: 5
  modified_base 35..44: mod_base = OTHER; note = internucleoside bonds are phosphorothioate bonds
  source 1..44: mol_type = other DNA

SEQ ID NO: 12 - DNA, 44 bp, synthetic construct; identical to SEQ ID NO: 6
  modified_base 1..9: mod_base = OTHER; note = internucleoside bonds are phosphorothioate bonds
  source 1..44: mol_type = other DNA

SEQ ID NO: 13 - DNA, 44 bp, synthetic construct; identical to SEQ ID NO: 7
  modified_base 36..44: mod_base = OTHER; note = internucleoside bonds are phosphorothioate bonds
  source 1..44: mol_type = other DNA

SEQ ID NO: 14 - protein, 522 aa, Homo sapiens
MQPWHGKAMQRASEAGATAPKASARNARGAPMDPTESPAAPEAALPKAGKFGPARKSGSRQKKSAPDTQERPPVRATGARAKKAPQRAQDTQPSDATSAPGAEGLEPPAAREPALSRAGSCRQRGARCSTKPRPPPGPWDVPSPGLPVSAPILVRRDAAPGASKLRAVLEKLKLSRDDISTAAGMVKGVVDHLLLRLKCDSAFRGVGLLNTGSYYEHVKISAPNEFDVMFKLEVPRIQLEEYSNTRAYYFVKFKRNPKENPLSQFLEGEILSASKMLSKFRKIIKEEINDIKDTDVIMKRKRGGSPAVTLLISEKISVDITLALESKSSWPASTQEGLRIQNWLSAKVRKQLRLKPFYLVPKHAKEGNGFQEETWRLSFSHIEKEILNNHGKSKTCCENKEEKCCRKDCLKLMKYLLEQLKERFKDKKHLDKFSSYHVKTAFFHVCTQNPQDSQWDRKDLGLCFDNCVTYFLQCLRTEKLENYFIPEFNLFSSNLIDKRSKEFLTKQIEYERNNEFPVFDEF

SEQ ID NO: 15 - DNA, 44 bp, synthetic construct
  source 1..44: mol_type = other DNA
  misc_difference 1: note = t is uracil or thymine
  misc_difference 7: note = t is uracil or thymine
  misc_difference 11: note = t is uracil or thymine
  misc_difference 13: note = t is uracil or thymine
  misc_difference 16: note = t is uracil or thymine
  misc_difference 21: note = t is uracil or thymine
  misc_difference 25: note = t is uracil or thymine
  misc_difference 29: note = t is uracil or thymine
  misc_difference 32: note = t is uracil or thymine
  misc_difference 36..37: note = t is uracil or thymine
  misc_difference 39: note = t is uracil or thymine
tcaacatcagtctgataagctaagtcagtcatagcttatcagac

SEQ ID NO: 16 - DNA, 44 bp, synthetic construct
  source 1..44: mol_type = other DNA
  misc_difference 1: note = t is uracil or thymine
  misc_difference 5..6: note = t is uracil or thymine
  misc_difference 8: note = t is uracil or thymine
  misc_difference 14: note = t is uracil or thymine
  misc_difference 17: note = t is uracil or thymine
  misc_difference 19..20: note = t is uracil or thymine
  misc_difference 24: note = t is uracil or thymine
  misc_difference 26: note = t is uracil or thymine
  misc_difference 29: note = t is uracil or thymine
  misc_difference 34: note = t is uracil or thymine
  misc_difference 36: note = t is uracil or thymine
  misc_difference 40: note = t is uracil or thymine
  misc_difference 44: note = t is uracil or thymine
tagcttatcagactgatgttgagtctgataagctatgactgact

SEQ ID NO: 17 - DNA, 44 bp, synthetic construct
  source 1..44: mol_type = other DNA
  misc_difference 1: note = t is uracil or thymine
  misc_difference 7: note = t is uracil or thymine
  misc_difference 11: note = t is uracil or thymine
  misc_difference 13: note = t is uracil or thymine
  misc_difference 16: note = t is uracil or thymine
  misc_difference 21: note = t is uracil or thymine
  misc_difference 26: note = t is uracil or thymine
  misc_difference 30..31: note = t is uracil or thymine
  misc_difference 35..36: note = t is uracil or thymine
  misc_difference 38: note = t is uracil or thymine
  misc_difference 44: note = t is uracil or thymine
tcaacatcagtctgataagctagactgacttagcttatcagact

SEQ ID NO: 18 - DNA, 44 bp, synthetic construct
  source 1..44: mol_type = other DNA
  misc_difference 1: note = t is uracil or thymine
  misc_difference 5..6: note = t is uracil or thymine
  misc_difference 8: note = t is uracil or thymine
  misc_difference 14: note = t is uracil or thymine
  misc_difference 17: note = t is uracil or thymine
  misc_difference 19..20: note = t is uracil or thymine
  misc_difference 25: note = t is uracil or thymine
  misc_difference 27: note = t is uracil or thymine
  misc_difference 30: note = t is uracil or thymine
  misc_difference 35: note = t is uracil or thymine
  misc_difference 39: note = t is uracil or thymine
  misc_difference 43: note = t is uracil or thymine
tagcttatcagactgatgttgaagtctgataagctaagtcagtc

SEQ ID NO: 19 - DNA, 44 bp, synthetic construct; identical to SEQ ID NO: 15
  modified_base 1..10: mod_base = OTHER; note = internucleoside bonds are phosphorothioate bonds
  source 1..44: mol_type = other DNA

SEQ ID NO: 20 - DNA, 44 bp, synthetic construct; identical to SEQ ID NO: 16
  modified_base 35..44: mod_base = OTHER; note = internucleoside bonds are phosphorothioate bonds
  source 1..44: mol_type = other DNA

SEQ ID NO: 21 - DNA, 44 bp, synthetic construct; identical to SEQ ID NO: 17
  modified_base 1..9: mod_base = OTHER; note = internucleoside bonds are phosphorothioate bonds
  source 1..44: mol_type = other DNA

SEQ ID NO: 22 - DNA, 44 bp, synthetic construct; identical to SEQ ID NO: 18
  modified_base 36..44: mod_base = OTHER; note = internucleoside bonds are phosphorothioate bonds
  source 1..44: mol_type = other DNA